(19) United States
(12) Patent Application Publication
(10) Pub. No.: US 2023/0218432 A1
(43) Pub. Date: Jul. 13, 2023

(54) SYSTEMS AND METHODS FOR THERMAL FIBER STIMULATION

(71) Applicant: Soovu Labs, Inc., Seattle, WA (US)

(72) Inventors: Charles Chabal, Seattle, WA (US); Allan H. Stephan, Seattle, WA (US); Peter J. Dunbar, Seattle, WA (US); Jack Profit, Seattle, WA (US)

(21) Appl. No.: 17/928,990

(22) PCT Filed: Jun. 2, 2021
(86) PCT No.: PCT/US2021/035536

§ 371 Date: Dec. 1, 2022

Related U.S. Application Data
(60) Provisional application No. 63/033,635, filed on Jun. 2, 2020.
(60) Provisional application No. 63/093,736, filed on Oct. 19, 2020.

(51) Int. Cl. A61F 7/12

(52) U.S. Cl. CPC A61F 7/12; A61F 2007/126

(57) ABSTRACT

Devices, systems, and methods for applying pulsed energy to effectuate a physiological response in a human subject. Systems and methods disclosed herein can include, for example, a stimulus device that applies pulsed energy, such as heat, into a volume of tissue. The stimulus device can include one or more electrodes and can be affixed to the skin or implanted at a target site within the body. The systems and method can further include a monitoring device that detects at least one physiological parameter of the human subject during application of the pulsed energy, such as blood flow, oxygen delivery, muscle tension, subcutaneous or muscle temperatures, or brain activity. A control device can use the detected physiological parameter to define treatment parameters of the stimulus device such that the pulsed energy synchronizes with the measured physiological parameter to effectuate a desired response, such as reducing pain, suppressing appetite, and/or activating a hedonic response.

### PRIORITY CLAIM

This application claims the benefit of U.S. Provisional Patent Application No. 63/033,635, entitled “SYSTEMS AND METHODS FOR IMPROVED PAIN RELIEF VIA THERMAL FIBER STIMULATION” filed on Jun. 2, 2020 and U.S. Provisional Patent Application No. 63/093,736, entitled “SYSTEMS AND METHODS FOR IMPROVED PAIN RELIEF VIA THERMAL FIBER STIMULATION” filed on Oct. 19, 2020, both of which are incorporated by reference herein in their entirety.

### TECHNICAL FIELD

The following disclosure relates generally to stimulus-based therapeutic devices, systems, and methods. In particular, the disclosure relates to systems and methods for applying heat, vibration, electrical, and other stimuli to a patient's body for therapeutic purposes.

### BACKGROUND

In 1965, Melzack and Wall described the physiologic mechanisms by which stimulation of large diameter non-pain sensory nerves could reduce the amount of unpleasant activity carried by pain nerves. This landmark observation published in Science was termed the “gate control theory” and offered a model to describe the interactions between various types of sensory pathways in the peripheral and central nervous systems. The model described how non-painful sensory input such as mild electrical stimulation could reduce or gate the amount of nociceptive (painful) input that reached the central nervous system.

The gate-control theory stimulated research that lead to the creation of new medical devices such as transcutaneous electrical nerve stimulators (TENS). In brief, TENS works by electrically “blocking” pain impulses carried by peripheral nerves. Receptors to cold and heat are located just below the surface of the skin. Heat receptors are activated through a temperature range of about 36° C. to 45° C. and cold receptors by a temperature range of about 1-20° C. below the normal skin temperature of 34° C. (Van Hees and Gybels, 1981). The stimuli are transmitted centrally by thin poly-modal C nerve fibers. Activation of heat receptors is also affected by the rate of rise of the heat stimuli (Yarnitsky, et al., 1992). Above 45° C. warm receptor discharge decreases and nociceptive response increases, producing the sensations of pain and burning (Torebjork et al., 1984).

Activation of poly-modal thermal receptors causes significant pain relief in controlled experimental conditions. Kakigi and Watanabe (1996) demonstrated that warming and cooling of the skin in human volunteers could significantly reduce the amount of reported pain and somatosensory evoked potential activity induced by the noxious stimulation of a CO₂ laser. The authors offered that the effects seen could be from a central inhibitory effect produced by the thermal stimulation. Similar inhibition of pain from thermal simulation was reported in a different human experimental pain model (Ward et al., 1996). The study authors (Kakigi and Watanabe 1996 and Ward et al., 1996) proposed that the thermal analgesia was in part from a central inhibitory effect (gating) from stimulation of small thin C nerve fibers. This contrasts with TENS which produces at least part of its analgesia through gating brought on by activation of large diameter afferent nerve fibers.

A number of recent clinical studies strongly support the use of heat as an analgesic in patients who suffer from chronic pain and offer potential mechanisms by which heat produces analgesia. Abeln et al. (2000) in a randomized controlled single-blinded study examined the effect of low-level topical heat in 76 subjects who suffered from low back pain. Heat treatment was statistically more effective in relieving pain and improving the quality of sleep than that produced by placebo.

Weingand et al. (2001) examined the effects in a randomized, single blinded, controlled trial of low-level topical heat in a group of over 200 subjects who suffered from low back pain and compared heat to placebo heat, an oral analgesic placebo, and ibuprofen 1200 mg/day. The authors found heat treatment more effective than placebo and superior to ibuprofen treatment in relieving pain and increasing physical function as assessed by physical examination and the Roland Morris disability scale.

A separate group (Nadler et al., 2002) found similar results in a prospective single blinded randomized controlled trial of 371 subjects who suffered from acute low back pain. The authors found that cutaneous heat treatment was more effective than oral ibuprofen 1200 mg/day, acetaminophen 4000 mg/day or oral and heat placebos in producing pain relief and improving physical function. The authors offered several hypotheses for the mechanism(s) of action which includes increased muscle relaxation, connective tissue elasticity, blood flow, and tissue healing potential provided through the low-level topical heat. Similar beneficial effects of topical heat were show in patients who suffered from dysmenorrhea (Akin et al., 2001), and temporomandibular joint pain TMJ (Nelson et al., 1988).

A recent study used power Doppler ultrasound to evaluate the effects of topical heat on muscle blood flow in humans (Erasala et al., 2001). Subjects underwent 30 minutes of heating over their trapezius muscle and changes in blood flow were examined at 18 different locations over the muscle. Vascularity increased 27% (p=0.25), 77% (p=0.03) and 104% (p=0.01) with 39° C., 40° C., or 42° C. temperature of the heating pad. Importantly, increases in blood flow extended approximately 3 cm deep into the muscle. The authors concluded that the increased blood flow likely contributed to the analgesic and muscle relaxation properties of the topical heat. Similar increases in deep vascular blood flow were noted using magnetic resonance thermometry in subjects treated with mild topical heat by two separate groups (Mulkern et al., 1999, and Reid et al., 1999) and using ultrasound and deep tissue thermistors (Petrofsky et al., 2016).

Recent studies demonstrated the analgesic effectiveness of heat and provided potential mechanisms of action. The mechanisms include a reduction of pain through a central nervous system interaction mediated via thin C-fibers (Kakigi and Watanabe, 1996, Ward et al. 1996), enhancement of superficial and deeper level blood flow (Erasala et al., 2001, Mulkern et al., 1999, Reid et al., 1999), or local effects on the muscle and connective tissue (Nadler et al., 2002, Akin et al. 2001). TENS is thought to act through inhibition of nociception by increasing endogenous opioids or by a neural inhibitory interaction of nociception via large diameter fibers. It is likely that TENS and heat act partly through different mechanisms with the potential for enhanced or even synergistic interactions. TENS is widely used and endorsed by the pain management guidelines of both the AHCPR and American Geriatric Society (Gloth 2001). However, a significant number of patients fail to achieve adequate relief with TENS or fail within six months of starting treatment (Fishbain et al., 1996).

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1A is a perspective view of a stimulus pod system configured in accordance with embodiments of the present technology.

FIG. 1B is an exploded view of a stimulus pod of the system shown in FIG. 1A configured in accordance with embodiments of the present technology.

FIG. 1C is an exploded view of an anchor of the system shown in FIG. 1A configured in accordance with embodiments of the present technology.

FIGS. 2A-2C are side cross-sectional views of the system shown in FIG. 1A illustrating a stimulus pod secured against an anchor in accordance with embodiments of the present technology.

FIGS. 3A-3C are enlarged side-cross sectional views of a portion of the system shown in FIG. 2A and illustrating various attachment mechanisms for attaching the stimulus pod to the anchor in accordance with embodiments of the present technology.

FIGS. 3D and 3E are enlarged side-cross sectional views of a portion of the system shown in FIG. 2B and illustrating various attachment mechanisms for attaching the stimulus pod to the anchor in accordance with embodiments of the present technology.

FIG. 3F is a bottom cross-sectional view of the system shown in FIG. 2C and illustrating an attachment mechanism for attaching the stimulus pod to the anchor in accordance with embodiments of the present technology.

FIG. 4 is a perspective view of a stimulus pod configured in accordance with embodiments of the present technology.

FIG. 5 is a schematic view of a stimulus delivery system configured in accordance with embodiments of the present technology.

FIG. 6 is a schematic view of a stimulus delivery system configured in accordance with embodiments of the present technology.

FIG. 7 illustrates a plurality of stimulus pods positioned on a back view of a human form in accordance embodiments of the present technology.

FIG. 8 is a graph of temperature versus time illustrating a variable heat cycle in accordance with embodiments of the present technology.

FIG. 9 is a graph of temperature versus time illustrating a variable heat cycle in accordance with embodiments of the present technology.

FIG. 10 is a graph of energy applied versus time illustrating the resultant skin temperature of a subject in accordance with embodiments of the present technology.

FIG. 11 is a graph of energy applied versus time in accordance with another embodiment of the present technology.

FIG. 12 illustrates energy applied to exemplary thermal zones and the resultant skin temperature of a subject in accordance with embodiments of the present technology.

FIG. 13 illustrates an on-demand pattern of variable heat cycles over time as requested by a subject in accordance with embodiments of the present technology.

FIG. 14 is a flow diagram illustrating a method of pulsing and cycling heat in accordance with embodiments of the present technology.

FIGS. 15A and 15B illustrate a hedonic device for activating the hedonic response configured in accordance with embodiments of the present technology.

FIGS. 16A and 16B illustrate flow diagrams of methods for activating the hedonic response of a subject in accordance with embodiments of the present technology.

FIGS. 17A and 17B illustrate flow diagrams of methods for suppressing appetite of a subject in accordance with embodiments of the present technology.

FIG. 18 illustrates a system for delivering a stimulus and monitoring physiological parameters of a patient configured in accordance with embodiments of the present technology.

FIG. 19 is a flow diagram illustrating the processing of a program component in accordance with some embodiments of the present technology.

FIG. 20 is a side view of a human form wearing a plurality of stimulus devices in accordance with select embodiments of the present technology.

FIG. 21 illustrates a flow diagram of a method for promoting pain relief and recovery around the joints and/or muscles of a human body in accordance with embodiments of the present technology.

FIG. 22 illustrates a flow diagram of a method for promoting pain relief and recovery around the joints and/or muscles of a human body in accordance with embodiments of the present technology.

FIGS. 23A and 23C are partially schematic views of rotating stimulus delivery systems configured in accordance with embodiments of the present technology.

FIGS. 23B and 23D illustrate rotating stimulus devices configured in accordance with embodiments of the present technology.

FIGS. 24A-24C are graphs illustrating energy application protocols for multiple stimulus elements in a rotating stimulus delivery system in accordance with embodiments of the present technology.

FIG. 25 is a flow diagram of a method of activating the stimulus delivery system in accordance with embodiments of the present technology.

FIG. 26 is a flow diagram of a method of activating the stimulus delivery system in accordance with embodiments of the present technology.

FIG. 27 is a flow diagram of a method of activating the stimulus delivery system in accordance with embodiments of the present technology.

FIG. 28 is a graph illustrating pain over time as a result of different heating protocols in accordance with embodiments of the present technology.

FIG. 29 is a front view of a pain relief device attached to a human subject and configured to project heat to underlying nerves in accordance with embodiments of the present technology.

FIG. 30 is a transverse view of implantable electrodes positioned in an arm of a human subject in accordance with embodiments of the present technology.

FIG. 31 is a transverse view of an implantable electrode positioned in an upper leg of a human subject in accordance with embodiments of the present technology.

FIG. 32A is a transverse view of electrodes implanted along a spinal cord of a human subject in accordance with embodiments of the present technology.

FIG. 32B is a longitudinal view of one of the electrodes of FIG. 32A implanted along the spinal cord of the human subject.

FIG. 33 is a transverse view of electrodes implanted along a spinal cord of a human subject in accordance with embodiments of the present technology.

### DETAILED DESCRIPTION

The present technology is directed generally to system, devices, and associated methods for applying stimuli, in particular pulsed heat, to various parts of the body of a human subject or patient and monitoring physiological and other parameters resulted from the applied stimuli.

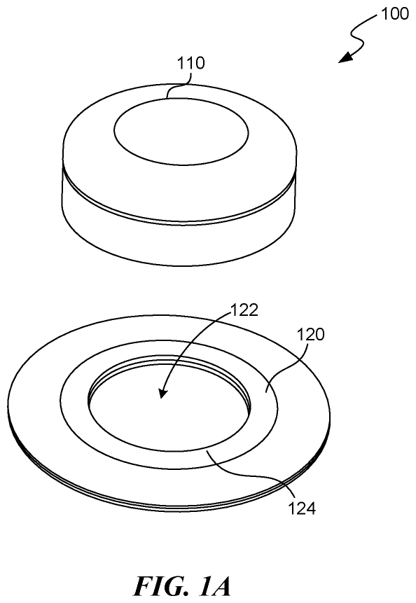

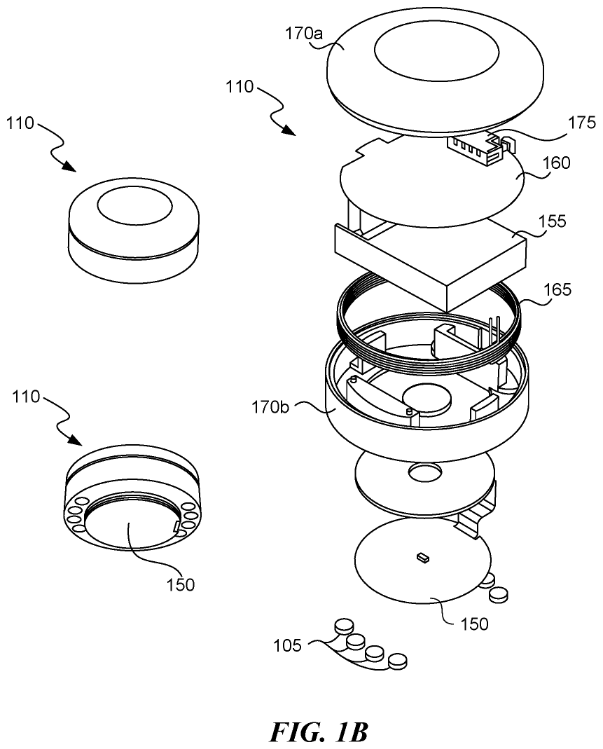

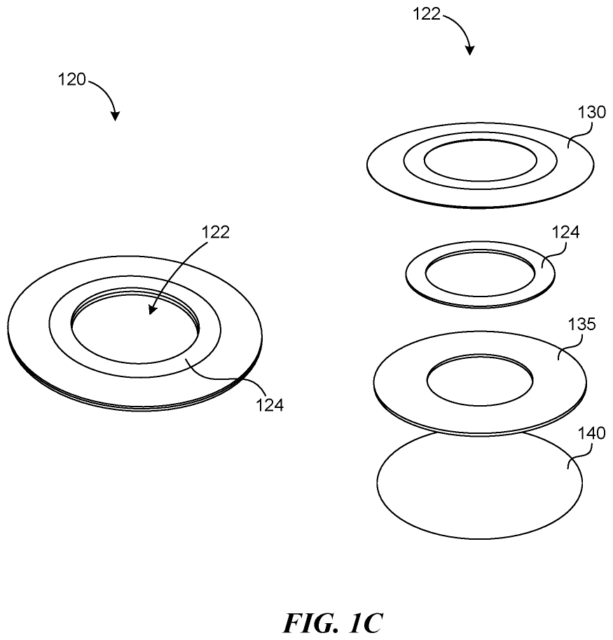

Several details describing thermal and electrical principles are not set forth in the following description to avoid unnecessarily obscuring embodiments of the present technology. Moreover, although the following disclosure sets forth several embodiments of the present technology, other embodiments can have different configurations, arrangements, and/or components than those described herein without departing from the spirit or scope of the present technology. For example, other embodiments may have additional elements, or they may lack one or more of the elements described in detail below with reference to FIGS. 1-33.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1-33.

Reference throughout this specification to “one embodiment” or “an embodiment” means that a particular feature, structure or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “substantially”, “approximately”, and “about” are used herein to mean the stated value plus or minus 10%.

The terms used herein are not intended—and should not be taken—to exclude from the scope of this present technology other types of heat sources that are designed to be placed on the skin to enable pain relief. Illustrative embodiments will be shown and described; however, one skilled in the art will recognize that the illustrative embodiments do not exclude other embodiments.

The headings provided herein are for convenience only and should not be construed as limiting the subject matter disclosed.

### I. SELECTED EMBODIMENTS OF STIMULUS POD SYSTEMS

FIG. 1A is a perspective view of a stimulus pod system 100 (“system 100”) configured in accordance with embodiments of the present technology. In the illustrated embodiment, the system 100 includes a stimulus pod 110 and an anchor 120. The stimulus pod 110 can be between about 0.5 inch (1.27 cm) to 2 inches (5.08 cm) in diameter (e.g., about 1 inch (2.54 cm) in diameter) and can be equipped to deliver different stimuli to a patient's body, including heat, vibration, and/or electricity. In some embodiments, the stimulus pod 110 can include sensors that gather information and relay the information back to a control station. The anchor 120 can have an adhesive surface that can be applied to various locations on the patient's body, an aperture 122, and an attachment ring 124 that can engage the stimulus pod 110 to hold the stimulus pod 110 onto the patient's body. Additionally, or alternatively, the stimulus pod 110 can be kept in place by clothing, magnets, a Velcro-type applicator, elastic bands, pocket-like holders, braces, or other type of applicators capable of holding the stimulus pod 110 against the patient's skin.

FIG. 1B is an exploded view of the pod 110 shown in FIG. 1A configured in accordance with embodiments of the present technology. In the illustrated embodiment, the stimulus pod 110 includes a stimulus surface 150 that contacts the patient's skin to deliver heat, mild electrical stimuli, vibration, and/or other stimuli to the patient's body in a measured, deliberate pattern to relieve pain and discomfort in the patient's body. The stimulus surface 150 may be about 3 square inches (19.35 cm²) or less. In some embodiments, the stimulus surface may be configured to transfer heat to the skin, and the stimulus surface material may be selected based on a desired thermal conductivity. For example, the stimulus surface may include a conductive heating element, a convective heating element, or a radiation-heating element. In another example, the heating surface may be an electrically resistive heating element with a low thermal mass to facilitate fast thermal rise on activation and fast thermal decay upon deactivation.

In the illustrated embodiment, the stimulus pod 110 includes a battery 155, a circuit board 160, a charging coil 165, and several housing elements 170 (individually referred to as an upper cover 170a and a body 170b). The battery 155 can power the stimulus surface and the circuit board 160. The battery 155 can be a lithium polymer battery or another suitable battery type. The charging coil 165 can be configured to receive power from a power source (e.g., a charging station) and deliver the power to the battery 155. In some embodiments, the stimulus pod 110 can include a wireless communication link 175 through which the stimulus pod 110 receives instructions and/or sends data to and from a control station. In some embodiments, the control station can be a mobile application contained on a cell phone. The housing elements 170 can enclose the internal components of the stimulus pod 110 and provide a convenient handling surface.

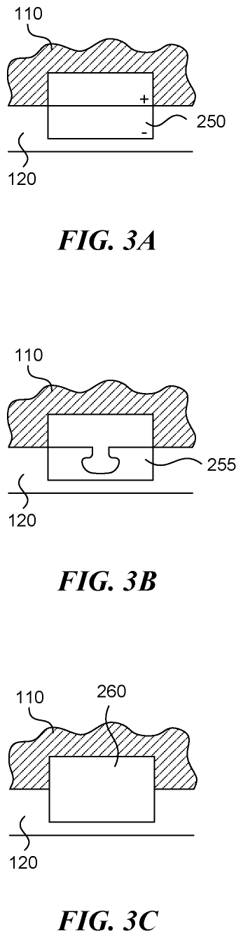

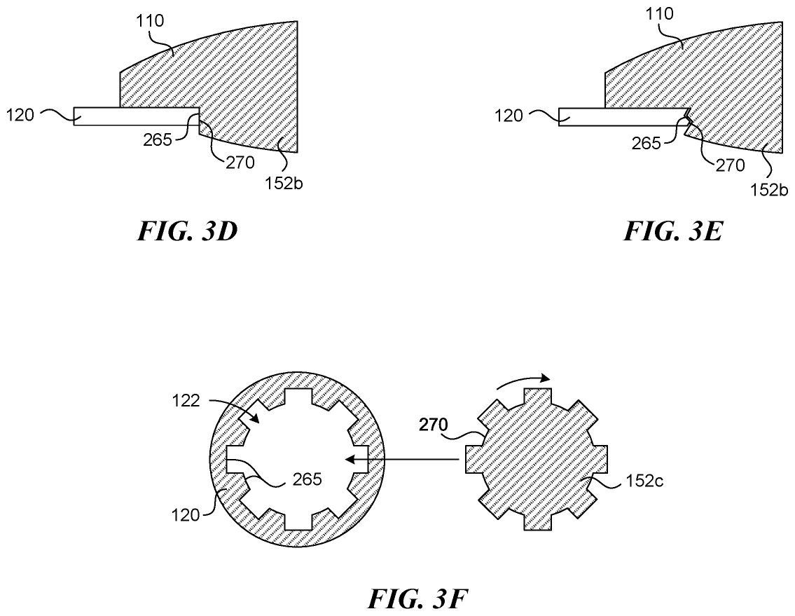

In some embodiments, as described in greater detail below with reference to FIGS. 3A-3F, the stimulus pod 110 can include attachment means to attach the stimulus pod 110 to the anchor 120. Referring to FIGS. 1A and 1B, for example, the stimulus pod 110 can have metal slugs 105 that can be magnetized and coupled to the attachment ring 124 (e.g., a metallic ring) in the anchor 120 to hold the stimulus pod 110 to the anchor 120. In some embodiments, the metal slugs 105 can also be used for stimulus delivery. In some embodiments, the metal slugs 105 can be positioned on a top side of the stimulus pods 110 and can be used to interface with a charging station and/or other external device.

In some embodiments, multiple ones of the stimulus pods 110 can be used in concert at different places on the patient's body. In some embodiments, the stimulus pods 110 can also be used to deliver medicine to a patient through electrophoresis, iontophoresis, and/or heat-enhanced perfusion due to capillary dilation. Electrophoresis is the motion of dispersed particles relative to a fluid under the influence of a spatially uniform electric field. Electrophoresis is ultimately caused by the presence of a charged interface between the particle surface and the surrounding fluid. Iontophoresis (a.k.a., Electromotive Drug Administration (EMDA)) is a technique using a small electric charge to deliver a medicine or other chemical through the skin. It is basically an injection without the needle. The technical description of this process is a non-invasive method of propelling high concentrations of a charged substance, normally a medication or bioactive agent, transdermally by repulsive electromotive force using a small electrical charge applied to an iontophoretic chamber containing a similarly charged active agent and its vehicle. One or two chambers are filled with a solution containing an active ingredient and its solvent, also called the vehicle. The positively charged chamber (anode) will repel a positively charged chemical, whereas the negatively charged chamber (cathode) will repel a negatively charged chemical into the skin.

FIG. 1C is an exploded view of the anchor shown in FIG. 1A configured in accordance with embodiments of the present technology. In the illustrated embodiment, the anchor 120 includes the attachment ring 124, an upper surface 130, an adhesive layer 135, and a liner 140. In some embodiments, the liner 140 can be removed to expose the adhesive layer 135 before placing the anchor 120 on the patient's body. The upper surface 130 is exposed to ambient conditions and accordingly can be similar to a bandage or a wound covering to provide a clean, water-resistant surface for the anchor 120. The attachment ring 124 is positioned beneath the upper surface 130 and can be a metallic ring, such as a steel ring, that can be coupled to the magnetic metal slugs 105 of the stimulus pod 110 and/or other components of the stimulus pod 110. The attachment ring 124 is held to the upper surface 130 by the adhesive layer 135, which can have an adhesive on (i) an upper side thereof to adhere to the attachment ring 124 and the upper surface 130 and (ii) on a lower side thereof to adhere to the liner 140. The lower side of the adhesive layer 135 may optionally include an applicator zone carrying an analgesic. The materials used to form the anchor 120 can all be rigid enough to maintain a proper shape, but flexible enough to substantially conform to the patient's body. For example, the attachment ring 124 can be segmented or thin to permit the anchor 120 to flex to some degree.

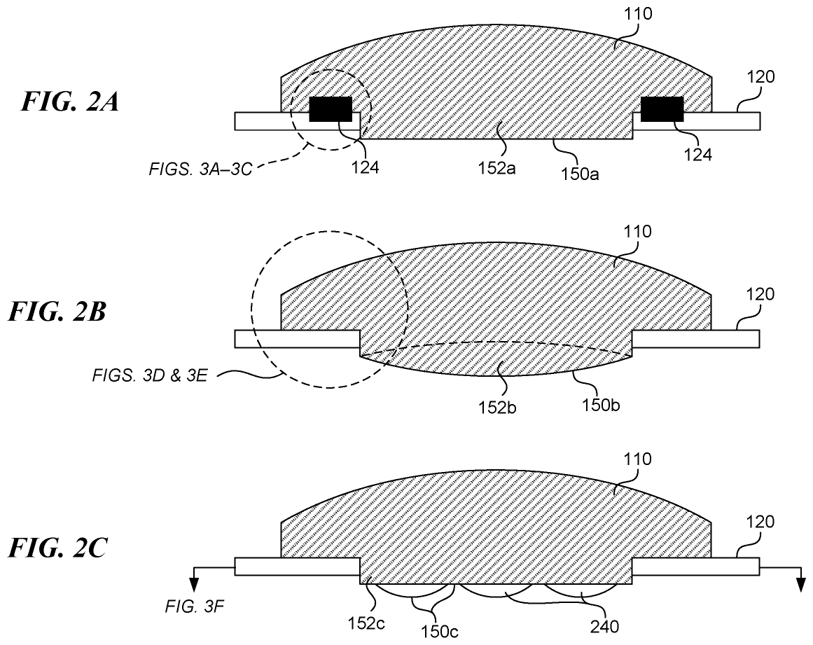

Referring to FIGS. 1A-1C together, once the anchor 120 is placed on the body of the patient (e.g., adhered to the body of the patient via the adhesive layer 135), the stimulus pod 110 can be placed into the aperture 122 in the anchor 120 and held in contact with the patient's body to deliver heat and/or other stimulation to the patient. Specifically, in many applications, the stimulus from the stimulus pod 110 can be delivered to the patient's body with the stimulus surface 150 directly contacting the patient's skin. Accordingly, at least a portion of the stimulus pod 110 can project beyond/through the anchor 120 such that the stimulus surface is in direct contact with the patient's skin. FIGS. 2A-2C, for example, are side cross-sectional views of the system 100 with the stimulus pod 110 secured against the anchor 120 and having various stimulus surfaces 150 (individually labeled as stimulus surfaces 150a-150c) in accordance with embodiments of the present technology.

Referring to FIG. 2A, in some embodiments the stimulus pod 110 can have a plug 152a that extends slightly beyond (e.g., projects past a lower surface of) the anchor 120. In the illustrated embodiment, the plug 152a has a stimulus surface 150a with a flat (e.g., generally planar) profile. The attachment ring 124 can engage the stimulus pod 110 with sufficient force such that the stimulus surface 150a presses down onto the patient's skin to ensure sufficient contact with the skin.

Referring to FIG. 2B, in some embodiments the stimulus pod 110 can have a plug 152b with a convex stimulus surface 150b that extends beyond the anchor 120. The slope of the convex stimulus surface 150b can depend in part on the size of the stimulus pod 110 and its intended application. For example, the slope can be selected such that substantially the entire stimulus surface 150b contacts the patient's skin (e.g., such that the slope of the stimulus surface 150b is not too extreme). Accordingly, the convex stimulus surface 150b can have relatively more surface area than the flat stimulus surface 150a shown in FIG. 2A. Therefore, in some embodiments the stimulus surface 150b can contact a relatively greater area of the skin of the patient than the flat stimulus surface 150a.

Referring to FIG. 2C, in some embodiments the stimulus pod 110 can have a plug 152c that extends beyond the anchor 120 and that has a stimulus surface 150c with several small bumps or projections 240. The dimensions of the stimulus surface 150c and the projections 240 can be selected to increase the surface area of the stimulus surface 150c that contacts the patient's skin without creating void spaces or air pockets between the projections 240 that might reduce effective heat transfer or delivery of other stimuli or drugs. In some embodiments, the projections 240 are not discrete, but are continuous and/or sinusoidal.

The stimulus pod 110 can be attached to (e.g., secured against, retained by, etc.) the anchor 120 such that the stimulus surface 150 is secured against the patient's skin. FIGS. 3A-3F, for example, illustrate various attachment mechanisms for attaching the stimulus pod 110 to the anchor 120 in accordance with embodiments of the present technology. More specifically, FIGS. 3A-3C are enlarged side-cross sectional views of a portion of the system 100 shown in FIG. 2A; FIGS. 3D and 3E are enlarged side-cross sectional views of a portion of the system 100 shown in FIG. 2B; and FIG. 3F is a bottom cross-sectional view of the system 100 shown in FIG. 2C taken along the line shown in FIG. 2C.

Referring to FIG. 3A, in some embodiments the anchor 120 can include a metallic or magnetic ring 250 that corresponds to a magnet 185 in the stimulus pod 110. The magnetic force between the ring 250 and the magnet 185 can hold/secure the stimulus pod 110 in place relative to the anchor 120.

Referring to FIG. 3B, in some embodiments the anchor 120 can be held/secured to the stimulus pod 110 by a mechanical fastener 255 such as a snap, or other similar mechanical attachment means. In the illustrated embodiment, the anchor 120 includes a resilient recession and the stimulus pod 110 includes a matching, resilient projection that, when pressed together, mechanically hold the stimulus pod 110 in place on the anchor 120b. In other embodiments, the anchor 120 can include a projection and the stimulus pod 110 can include a mating recession. In some embodiments, the attachment mechanism at the interface between the anchor 120 and the stimulus pod 110 can operate along the same principle as a plastic cap on a cardboard cup, such as a coffee cup and lid.

Referring to FIG. 3C, in some embodiments the anchor 120 can be held/secured to the stimulus pod 110 by a hook-and-loop fastener 260.

Referring to FIG. 3D, in some embodiments the anchor 120 can include an interior surface 265 that engages an exterior surface 270 of the plug 152b of the stimulus pod 110 to secure the stimulus pod 110 to the anchor 120. More particularly, one or both of the surfaces 265, 270 can be formed of a resilient material such that, when the plug 152b is pressed into the aperture 122 in the anchor 120, the plug 152b snaps into place.

Referring to FIG. 3E, in some embodiments the surfaces 265, 270 can have corresponding/mating threads such that the stimulus pod 110 can be screwed into the anchor 120.

Referring to FIG. 3F, in some embodiments the interior surface 265 of the anchor can have a keyed, regular, irregular, or other pattern, and the exterior surface 270 of the stimulus pod 110 can include a corresponding/matching pattern configured to engage with the anchor 120 to hold the stimulus pod 110 in place.

Any of the attachment mechanisms illustrated in FIGS. 3A-3F provide a simple way for the patient to apply a stimulus pod 110 to their body. As one of ordinary skill in the art will appreciate, the various configurations of the anchor 120 and stimulus pod 110 shown in FIGS. 2A-3F can be combined and/or integrated together (e.g., to include a magnetic and friction-fit connection).

During operation of the system 100, multiple ones of the stimulus pods 110 can be interchanged between different ones of the anchors 120, and vice versa. A patient can use a stimulus pod 110 until the battery is depleted, and then simply swap in another stimulus pod 110 with a fresh battery. The attachment means can be strong enough and the dimensions of the stimulus pod 110 can be small enough that the stimulus pod 110 can be worn under the patient's clothing easily. The placement of the anchors 120 can vary greatly according to a predetermined diagnostic pattern or personal preference. In some embodiments, one or more of the stimulus pods 110 can be placed at an area of discomfort, such as a painful lower back. Some research suggests that placing additional stimulus pods 110 at an area remote from a problem area can also provide analgesic effects. For example, a patient may place one of the stimulus pods 110 at the lower back—where their pain is—but they can also use a second one of the stimulus pods 110 near the shoulders or on the legs. Multiple stimulus pods 110 can be used in concert to produce an aggregate affect. Because different areas of the human body have different nerve densities, in certain areas two of the stimulus pods 110 placed near one another can be perceived as a single, large stimulus pod. For example, the patient's back has much lower nerve density than the face, neck, or arms. Accordingly, the patient can use a pair of small stimulus pods 110 (e.g., one or two inches, or 2.54 or 5.08 cm, in diameter) at the lower back and spaced about three inches (7.62 cm) or four inches (10.16 cm) apart to achieve the same sensory result as a larger stimulus pod covering the entire area. An unexpected benefit of this arrangement is that much less power is required to provide the stimulus in two small areas than would be required to stimulate the entire area.

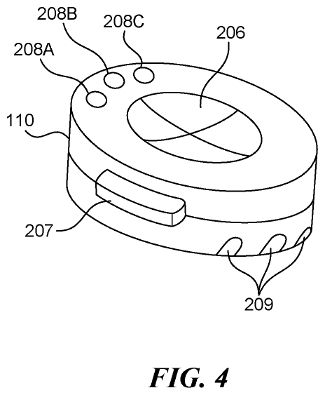

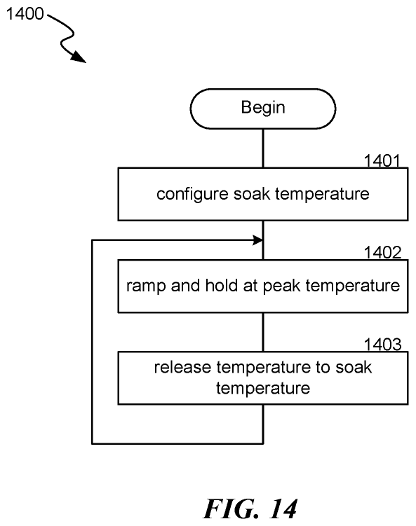

FIG. 4 is a perspective view of the stimulus pod 110 and shows additional features of the stimulus pod 110 in accordance with embodiments of the present technology. In the illustrated embodiment, the stimulus pod 110 has contacts 209 for interfacing with a charging station and that are positioned on a lower surface of the stimulus pod 110. In other embodiments, the contacts 209 can be positioned on an upper surface of the stimulus pod 110 or elsewhere on the stimulus pod 110. In the illustrated embodiment, the stimulus pod 110 further includes an on/off switch 207 for powering/de-powering the stimulus pod 110. Although a simple push-type on/off switch is illustrated, in other embodiments, the stimulus pod 110 can include other types of switches including, for example, a slide switch, an optical switch, a touch sensor, an accelerometer to detect tapping, etc. In use, the on/off switch 207 is typically activated after contact with the patient's skin has been established. In addition to its power on/off function, the on/off switch 207 can be configured to control a number of heat cycles and/or a temperature of the stimulus pod 110 (e.g., as described in detail below with reference to FIGS. 14-19).

In the illustrated embodiment, the stimulus pod 110 also includes a stimulus cycle switch 206 configured to, for example, switch between different levels of an applied stimulus (e.g., a low, medium, or high temperature). The stimulus pod 110 can also include indicators 208A-C such as LEDs that can light up in response to a particular setting of the stimulus cycle switch 206. In other embodiments, a single indicator 208 capable of changing its color, intensity, or other property can be used to indicate different settings of the stimulus pod 110. A push-type stimulus cycle switch 206 is illustrated in FIG. 4; however, in other embodiments other types of switches can be used such as, for example, a slide switch, multi-pole throw switch, touch sensitive switch, etc.

The stimulus pod 110 may also include an electrical circuit and a temperature measuring element configured to monitor the temperature of the skin. The temperature measuring element may be positioned, for example, in the center of the stimulus surface 150. The temperature measuring element may be operatively coupled to the electrical circuit, and the electrical circuit may be communicatively coupled to a monitoring/control device such as a desktop or laptop computer, a smartphone, a tablet, or other device. The electrical circuit may sense, via the temperature measuring element, a temperature of the skin and, based off the sensed temperature and/or other information, determine one or more characteristics of the skin. The electrical circuit may then transmit information about the one or more characteristics to the monitoring/control device, and the monitoring/control device may display the information to a user or health professional. For example, the electrical circuit may determine information about the skin's thermal transfer capacity and/or the skin's blood flow and send this information to the monitoring/control device for display. The electrical circuit may also utilize a look-up table, formula, chart, or other source of information to predict when thermal injury to the skin may occur. When the electrical circuit determines thermal injury may occur, the electrical circuit may instruct the monitoring/control device to provide a warning to the user, and/or may automatically turn the stimulus pod 110 off.

### II. SELECTED EMBODIMENTS OF STIMULUS DELIVERY SYSTEMS

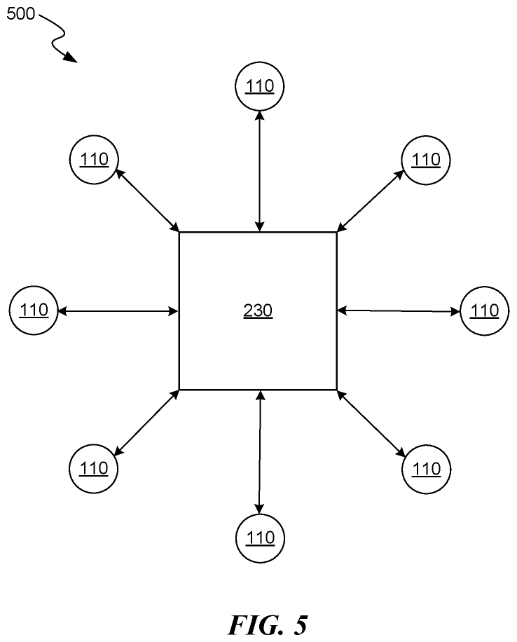

In some embodiments, one or more of the stimulus pods 110 can communicate with a control station to, for example, coordinate the delivery of stimulation to a patient at one or multiple locations. FIG. 5, for example, is a schematic view of a stimulus delivery system 500 configured in accordance with embodiments of the present technology. In the illustrated embodiment, the stimulus delivery system 500 includes one or more of the stimulus pods 110 communicatively coupled to a control station 230 (shown schematically in FIG. 5). The stimulus pods 110 can communicate with the control station 230 through any accepted wireless or wired protocol, including radio frequency (RF), infrared light, laser light, visible light, acoustic energy, BLUETOOTH, WIFI, or other communication systems. Additionally, the signals can be sent and received through the patient's skin. In addition to providing a communication path among the stimulus pods 110, sending and receiving signals through the patient's skin may be particularly well suited for determining a distance between the stimulus pods 110.

The control station 230 can be a desktop or laptop computer, a smartphone, a tablet, or other device. In some embodiments, the control station 230 can be included with or integrated into a charging station, and/or can share components such as a power source, circuitry, etc., with a charging station. The control station 230 can instruct one or more of the stimulus pods 110 to apply heat, electric stimuli, vibration, or other stimulus or combination of stimuli in various patterns to the patient's body. In other embodiments, the pods 110 include a button or series of buttons through which the pods 110 can be manually operated. The possible applications are many, and include various combinations of ramp up operations, maximum intensity operations (e.g., maximum temperature or maximum electrical current, etc.), ramp down operations, stimulus soak operations, and lockout period operations (e.g., as described in detail below with reference to FIGS. 14-19). In some embodiments, stimulus can be applied from different stimulus pods 110 at different levels and/or in different patterns. For example, a patient may place one of the stimulus pods 110 at their upper back, their lower back, and near each of their shoulders or in a different arrangement. The control station 230 can vary the stimulus application at the various zones according to a predetermined pattern. If a smartphone or other device having a screen is used as the control station 230, the screen may display a graphical representation of the patient's body with indication as to where to locate the pods 110 in a particular application. Furthermore, the screen may display countdown time information for all or some of the stimulus pods 110, and/or a battery status of the stimulus pods 110.

In several embodiments, the control station 230 can detect or receive information regarding the location of the stimulus pods 110 on the patient's body, and can vary the stimulus pattern accordingly. In one embodiment, the stimulus pods 110 can be built with certain body positions in mind. In some embodiments, the stimulus pods 110 can carry body position labels to instruct the patient to apply the stimulus pods 110 according to the label. For example, in a set of four stimulus pods, two can be marked “shoulders,” a third can be marked “lower back,” and a fourth can be marked “upper back.” In some embodiments, the anchors 120 can communicate their location to the stimulus pod 110. For example, the anchors 120 can include passive identifiers such as RFID tags or other simple, passive devices for communicating with the stimulus pods 110 and/or the control station 230. In such embodiments, the anchors 120 can remain in place even when different stimulus pods 110 are swapped in and out of the anchors 120. Therefore, the stationary anchors 120 can accurately provide location information to the control station 230 independent of which specific ones of the stimulus pods 110 occupy the anchors 120.

In other embodiments, the patient can inform the control station 230 where the stimulus pods 110 are situated, and with this information the control station 230 can apply the desired stimulus pattern to the stimulus pods 110. For example, the stimulus pods 110 can fire sequentially, and the patient can indicate the location of the stimulus on a user interface. Through the user interface, the patient can also operate the system 100 and apply treatment. In some embodiments, the control station 230 can graphically display a depiction of the patient's body, and the patient can indicate to the control station 230 where the stimulus pods 110 are located on their body. Alternatively, the patient can directly control the stimulus application through the stimulus pods 110 by moving a pointing device along the graphical depiction of their body to create a virtual stimulus-massage that the patient, or a healthcare professional, controls directly. In some embodiments, the control station 230 can include a touch screen that the patient can touch to apply heat or other stimulus to various portions of their body (or to the body of another patient).

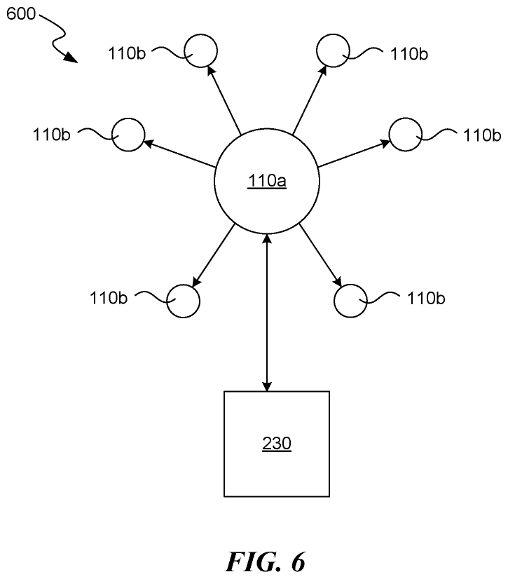

FIG. 6 is a schematic view of a stimulus delivery system 600 configured in accordance with embodiments of the present technology. In the illustrated embodiment, the stimulus delivery system 600 includes a plurality of the stimulus pods 110 communicatively coupled to the control station 230 (shown schematically in FIG. 6). At least one of the stimulus pods 110 can be configured as an index pod 110a, and the other ones of the stimulus pods 110 can be configured as dummy pods 110b. In some embodiments, the relationship between the index pod 110a and the dummy pods 110b can be similar to a master/drone relationship. For example, the index pod 110a can include more sophisticated telemetry equipment than the dummy pods 110b, and can act as an intermediary between the dummy pods 110b and the control station 230. The index pod 110a may include stimulus components, such as a heating surface or vibration equipment, and can deliver stimulus just like the dummy pods 110b. Alternately, the index pod 110a can be a dedicated index pod 110a with communication equipment, but without stimulus equipment.

In some embodiments, the index pod 110a and the control station 230 can discern when two or more of the stimulus pods 110 (e.g., dummy pods 110b or index pods 110a) are near enough to one another that they can work in aggregate. If the control station 230 knows where the stimulus pods 110 are placed on the patient's body, the control station 230, through the index pod 110a, can vary the threshold distance between the stimulus pods 110 as a function of nerve density at different locations on the body. For example, if the control station 230 discerns that two or more of the stimulus pods 110 are three inches (7.62 cm) apart and on the lower back, the control station 230 can operate those ones of the stimulus pods 110 together to effectively cover the area between the stimulus pods 110 as well as the area directly contacting the stimulus pods 110. By comparison, if two or more of the stimulus pods 110 are three inches (7.62 cm) apart, but are placed on a more sensitive area, such as the patient's face or neck, the control station 230 can determine that the aggregate effect may not be perceived to reach the area between those ones of the stimulus pods 110 because of the greater nerve density. This information can be used when applying a treatment plan that calls for stimulus on a prescribed area. In some embodiments, the control station 230 can determine whether one of the stimulus pods 110 is on or near the prescribed area, and if not, whether the aggregate effect from two or more of the stimulus pods 110 can be used to carry out the treatment plan and can execute the plan through the stimulus pods 110.

### III. SELECTED EMBODIMENTS OF METHODS OF APPLYING STIMULI TO DESENSITIZE THERMORECEPTORS

The present technology includes methods of applying stimuli to reduce pain. For example, certain methods described herein activate the skin's thermoreceptors (e.g., the thermoTRPs) to block and/or otherwise mask the sensation of pain. In some embodiments, the present technology is configured to desensitize specific thermoreceptors such as TRPV1, TRPV2, TRPV3, TRPV4, TRPA1, and/or TRPM8 to reduce the sensation of pain.

In some embodiments, both cold and warm thermoreceptors are targeted. In other embodiments, only cold receptors or only warm receptors are targeted. The present technology and methods may be advantageous over existing pain relief pharmaceutical formulations or devices because they locally target pain producing regions of the body and do not contain risks inherent with chronic pharmaceutical dosing. Moreover, the present technology and methods may be advantageous over existing thermal therapies because the present technology may recruit and desensitize more thermoreceptors, thus providing a more effective therapy.

The present technology includes applying a stimulus to the skin of a patient. A number of different stimuli may be utilized. For example, heat may be applied at any temperature in the range of about 35-49° C. For example, heat may be applied at about 35° C., 36° C., 37° C., 38° C., 39° C., 40° C., 41° C., 42° C., 43° C., 44° C., 45° C., 46° C., 47° C., 48° C., and/or 49° C. As discussed above, both cold and warm receptors (e.g., TRPM8 and/or TRPV1) may be activated by such temperatures, thereby desensitizing the receptors and contributing to pain reduction. Other suitable stimuli include compounds configured to activate cold receptors, warm receptors, and/or both cold and warm receptors. Such compounds may include, for example, menthol, menthol derivatives, icilin, capsaicin, and capsaicin derivatives.

In some embodiments, the stimulus is applied to one or more continuous surface areas of skin. For example, the stimulus may be applied to at least one surface area of skin that is 10 square inches (64.52 cm²) or less; the stimulus may be applied to at least one surface area of skin that is less than about 6 square inches (38.71 cm²); the stimulus may be applied to at least one surface area of skin that is less than about 3 square inches (19.35 cm²); and/or the stimulus may be applied to at least one surface area of skin that is less than about 2 square inches (12.9 cm²). In other embodiments, the stimulus may be applied to a surface area of skin greater than 10 square inches (64.52 cm²).

In some embodiments, the stimulus delivery systems described herein can be used to apply the therapeutic stimuli to a patient. The stimulus delivery systems may apply the stimuli in many different patterns, magnitudes, cycles, etc. For example, a control unit (e.g., the control station 230) can be used to activate and control one or more wearable devices (e.g., the stimulus pods 110) to apply stimuli according to a predetermined heating cycle and/or pattern. In some embodiments, the stimulus pods 110 are configured to be placed in various locations on the skin of the patient to provide therapeutic heat treatment for relieving pain. The following disclosure details a few specific methods of applying stimuli using the delivery systems of the present technology. However, one skilled in the art will appreciate that the present technology can be used in many different manners to alleviate pain, treat ailments, etc., without deviating from the scope of the present technology. Moreover, while reference is made herein to the stimulus pod system 100, one skilled in the art will appreciate that the following methods can be carried out using other suitable heat producing devices and/or topical compounds—for example, those described in detail in U.S. Pat. No. 7,871,427, tilted “APPARATUS AND METHOD FOR USING A PORTABLE THERMAL DEVICE TO REDUCE ACCOMMODATION OF NERVE RECEPTORS,” and filed Feb. 8, 2006, and/or U.S. Pat. No. 8,579,953, titled “DEVICES AND METHODS FOR THERAPEUTIC HEAT TREATMENT,” and filed Dec. 8, 2008, each of which is incorporated herein by reference in its entirety.

1. Selected Embodiments of Low-Level Heating and Cooling Combined with Intermittent High-Level Heating

In some embodiments, the present technology can be configured to apply a continuous amount of low-level heat combined with discrete amounts or intermittent bursts of high-level heat to a patient. The intermittent bursts of high-level heat may enable recruitment, activation, and/or desensitization of thermoreceptors that are normally only activated at temperatures higher than those provided by traditional heat therapy. For example, as discussed above, tissue damage may occur following prolonged exposure to temperatures above 40° C. Accordingly, traditional heat therapies typically do not exceed 40° C. However, the present technology can apply heat therapy at temperatures above 40° C. because the present technology can be configured to apply intermittent bursts of heat above 40° C. while avoiding the prolonged exposure that may lead to tissue damage. Because heat can be applied at temperatures above 40° C., more thermoreceptors may be recruited, activated, and/or desensitized than in traditional static heat therapy.

As described in detail below, the bursts of heat can be at distinct locations within or around the areas producing the low-level heat. The low-level heat can be maintained as a constant application of heat (e.g., heating below 42° C., 41° C., 40° C., etc.) while the high-level heat is applied in intermittent bursts (e.g., milliseconds in some embodiments). In certain embodiments of the present disclosure, bursts of heat in the range of about 40-55° C. are applied to discrete areas of skin to excite the receptors. Other suitable ranges may include, for example, about 40-49° C., 40-48° C., 40-47° C., 40-46° C., and/or 40-45° C. In other embodiments, however, the thermal bursts can include temperatures higher or lower than the range of 40-55° C. For example, the thermal bursts can include temperatures greater than 45° C. For example, the thermal bursts can include temperatures between about 45-60° C. In a further example, the thermal bursts can include temperatures between about 56-60° C. For the purposes of this disclosure, thermal bursts can be defined as the application of increased heat in discrete areas where the temperature of the burst ranges from 0.1° C. to 25° C. or more above the baseline temperature of the continuous low-level heat application. The thermal bursts can include a ramp up speed ranging from milliseconds to minutes to reach a maximum temperature. In addition, and as described below, the size of the area applying the thermal burst may be relatively small in comparison to the area applying the low-level heat. In other embodiments, however, the area applying the thermal burst may be approximately equal to or less than the area applying the low-level heat.

In some embodiments, a method of applying heat to a living body includes applying a constant amount of heat to a region of the body at a first temperature (e.g., via a stimulus pod 110). The method can also include applying intermittent amounts of heat to the region (e.g., via the stimulus pod 110). The intermittent amounts of heat may be applied at a second temperature greater than the first temperature.

In some embodiments, a method of applying heat to a living body includes applying a constant amount of heat to a first region of the body at a first temperature (e.g., via a first one of the stimulus pods 110). The method can also include applying intermittent amounts of heat to a second region of the body (e.g., via a second one of the stimulus pods 110). The intermittent amounts of heat may be applied at a second temperature greater than the first temperature. In some embodiments, the second region can partially or fully overlap the first region. And in some embodiments, the intermittent amounts of heat are delivered at pre-selected, focused points wherein the surface area of the second region is smaller than the surface area of the first region.

A method configured in accordance with another embodiment of the disclosure includes a method of exciting thermoreceptors in a living organism. The method includes heating a first portion of skin with a generally constant amount of heat at a baseline temperature (e.g., via a first one of the stimulus pods 110), and heating a second portion of skin with a burst of heat at a temperature above the baseline temperature (e.g., via a second one of the stimulus pods 110) while heating the first portion of skin with the generally constant amount of heat.

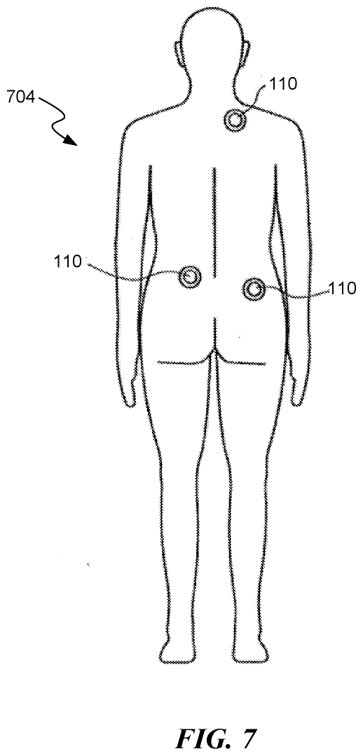

As described herein, certain methods in accordance with the present technology may utilize the stimulus pods 110. FIG. 7 illustrates a plurality of stimulus pods 110 positioned on a back view of a human form 704 (e.g., at the shoulder, lower back, and hip) in accordance with embodiments of the present technology. The stimulus pods 110 can be configured to provide continuous low-level heating with periodic bursts or impulses of high-level heat and can be applied simultaneously to various areas of the body 704 and can be used in conjunction with one another or independently to provide pain relief. Therefore, the stimulus pods 110 can accommodate users who suffer from pain in areas located in more than one region of the body thus requiring simultaneous treatment. For example, the treatment of conditions such as fibromyalgia, dysmenorrhea, PMS, back and neck pain, sports related injuries, chronic pain etc., may greatly benefit from locating the stimulus pods 110 at different positions to simultaneously treat one or more painful areas.

The combination of the continuous low-level heating and intermittent high-level heating at discrete, focused regions provides several advantages over conventional heating systems. The augmentation of the continuous heating (or cooling), for example, provides enhanced pain relief by promoting blood flow, increasing flexibility, and relaxing muscles, ligaments, and other tissues. The illustrated configuration achieves enhanced pain relief by providing a strong stimulation of the thermoreceptors in the skin and subcutaneous tissues of the body by rapidly changing temperatures. Both the rapid change in temperature (e.g., the rapid increase in temperature during the intermittent burst) and the ability to stimulate at higher temperatures (e.g., above about 40° C.) recruits more thermoreceptors, including TRPV1 and/or TRPM8. Accordingly, the intermittent focused bursts of heat, combined with the constant heat, provide for better receptor recruitment and stimulation, thereby leading to increased desensitization, resulting in better analgesic results.

2. Selected Embodiments of Heat Cycling to Desensitize Thermoreceptors

In some embodiments, the present technology can be used to provide energy and/or heat to a patient to desensitize certain pain-associated thermoreceptors (e.g., TRPV1). The method includes increasing the temperature of a heating element (e.g., one or more of the stimulus surfaces 150 of the stimulus pods 110) to provide a first temperature ramp-up period, holding the temperature of the heating element at a predetermined therapeutic level, decreasing the temperature of the heating device during a ramp-down period, and holding the temperature of the heating device at a predetermined soak level, wherein the soak level temperature is less than the therapeutic level temperature by at least 1° C.

In operation, the heating device (e.g., one or more of the stimulus pods 110) may deliver heat intermittently. The heat may be applied for a period long enough to heat the skin to a desired level; upon reaching the desired skin temperature the device turns off and the skin is allowed to cool; after a preprogrammed interval the device may reactivate the heat unit and the cycle repeats. Alternatively, multiple cycles may be delivered sequentially for a predetermined duration.

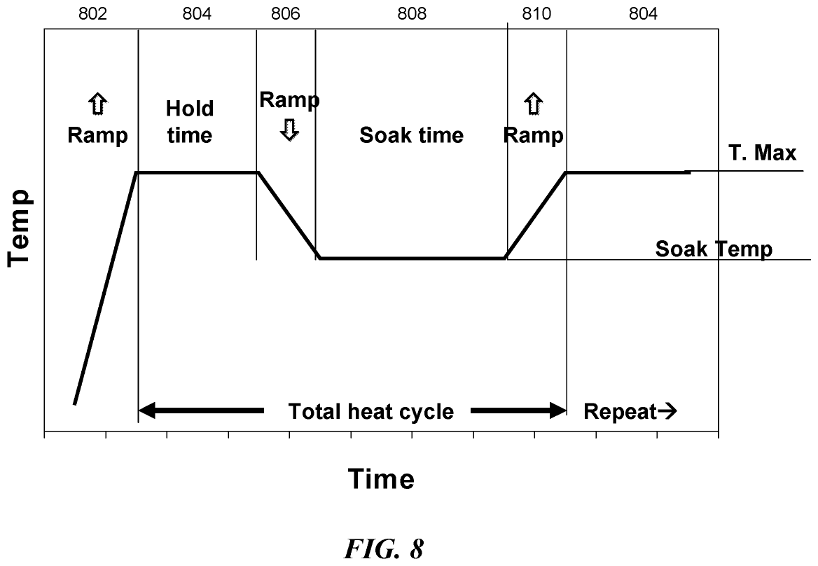

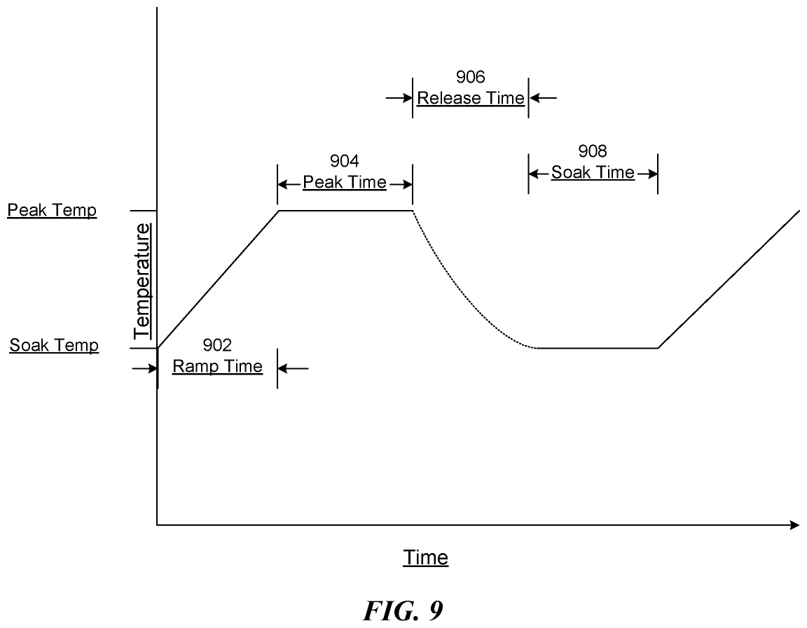

FIG. 8, for example, is a graph of temperature versus time illustrating a variable heat cycle for a heating element configured in accordance with embodiments of the present technology. In the illustrated embodiment, the variable heat cycle includes a first temperature ramp-up phase 802, a therapeutic temperature hold phase 804, a ramp-down phase 806, a soak phase 808, and a second ramp-up phase 810. FIG. 9 is a graph of temperature versus time illustrating a variable heat cycle for the heating element configured in accordance with embodiments of the present technology. In the illustrated embodiment, the variable heat cycle includes a ramp-up phase 902, a peak-time hold phase 904, a release phase 906, and a soak phase 908.

In some embodiments, the ramp-up phases 802, 810, and 902 may be less than about 4 seconds. For example, the ramp-up phase may be about 3 seconds, about 2 seconds, about 1 second, or less than 1 second. A short ramp up phase can be beneficial because a quick change in temperature (e.g., a short ramp-up phase) can recruit more thermoreceptors then a slow change in temperature. More specifically, a quick change in temperature can result in a rapid energy transfer between the heating device and the skin. This rapid energy transfer between the heating device and skin can activate thermoreceptors that may only traditionally be activated at higher static temperatures. Thus, more receptors may be recruited by “shocking” the receptors with a quick energy change. In contrast, a slower change in temperature (e.g., a ramp-up phase of about 4 seconds or more) provides a less intense change in energy and/or transfer of energy between the heating device and the skin and thus may not provide the same level of thermoreceptor recruitment. Accordingly, one benefit of the present technology is the recruitment of additional thermoreceptors by having a rapid energy transfer between the heating device and skin.

In some embodiments, the therapeutic temperature hold phase 804 and the peak-time hold phase 904 may be less than about 15 seconds. For example, the hold phases 1404 and 1504 may be about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, or about 15 seconds. The temperature during hold phases 804 and 904 may be greater than about 40° C. For example, the temperature during hold phases 804 and 904 may be defined within a range, such as between 40-49° C., 40-48° C., 40-47° C., 40-46° C., or 40-45° C., 41-45° C., 42-45° C., 43-45° C., 44-45° C., or any other suitable range between an upper bound of about 49° C. and a lower bound of about 40° C. In another example, the temperature during hold phases 804 and 904 may be defined within a range between an upper bound of about 60° C. and a lower bound of 50° C. The temperature during hold phases 804 and 904 may also be defined as a specific temperature, such as about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C. Additionally, or alternatively, the temperature during the hold phases 804 and 904 can be defined as a target skin temperature for a region of skin adjacent to the heating device. For example, the target skin temperature may be about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C. The target skin temperature may also be defined as a range of temperatures between, for example, 40-49° C., 40-48° C., 40-47° C., 40-46° C., or 40-45° C., 41-45° C., 42-45° C., 43-45° C., 44-45° C., 50-52° C., 53-55° C., 55-58° C., or 58-60° C. In some embodiments, the temperature may alternatively be defined as an energy applied to the skin.

The ramp-down phase 806 and the release phase 906 may take several different forms. For example, the ramp-down phase may be actively controlled and take a linear form, such as illustrated by ramp-down phase 806. The ramp-down phase may also simply be the result of turning off the energy or heat producing element such that a non-linear decay of heat occurs. For example, release phase 906 illustrates one such example of the non-linear decay of heat. The decay time will depend on a number of factors, including peak temperature, soak temperature, and the thermal conductivity of the heating surface.

In some embodiments, the soak phase 808 and 908 may be held at a temperature higher than the basal body temperature of the user, thus allowing continued therapeutic effects by improving the blood flow to the region and providing muscle relaxation. In other embodiments, the heating device may simply be turned off during the soak phase 808 and 908, such that the temperature of the heating device is near the room temperature and/or the basal body temperature of the user (assuming complete decay). The duration of the soak phase 808 and 908 can be selected to maintain desensitization of previously desensitized thermoreceptors while simultaneously keeping a thermal flux value within the skin below a tissue-damage inducing threshold. For example, the soak time may be optimized based on identifying a duration for a thermoreceptor to reset (e.g., recover from the CA²⁺ influx and be capable of firing again). In some embodiments, for example, the soak time could be 60 seconds or less, such as about 55 seconds, about 50 seconds, about 45 seconds, about 40 seconds, about 35 seconds, about 30 seconds, about 25 seconds, about 20 seconds, about 15 seconds, about 10 seconds, or about 5 seconds. In some embodiments, the soak time may be greater than 60 seconds or less than 5 seconds. In some embodiments, the soak time may be less than 120 minutes and greater than 1 second. For example, the soak time may be about 1 second, about 2 seconds, about 3 seconds, about 4 seconds, about 90 seconds, about 2 minutes, about 10 minutes, about 30 minutes, about 60 minutes, or about 120 minutes.

The heating cycle including the ramp-up phase, the hold phase, the ramp-down phase, and the soak phase may be repeated at a frequency to maintain at least a partial desensitization of certain thermoreceptors (e.g., TRPV1). Moreover, the heating cycle may be repeated continuously for a preset duration (e.g., five minutes) or number of cycles (e.g., 100 cycles). In some embodiments, the heating cycle may continue until turned off. In some embodiments, the ramp-up phase, the hold phase, the ramp-down phase, and the soak phase may be repeated at regular, irregular, and/or random intervals.

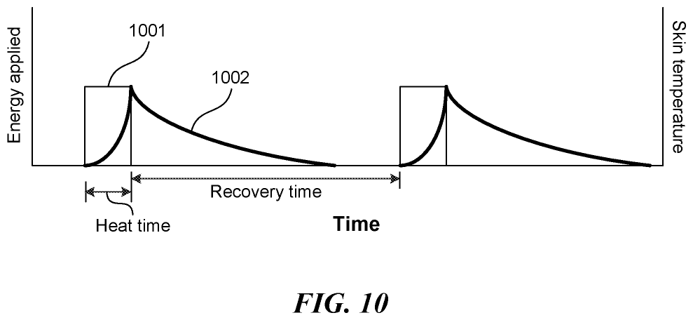

FIGS. 10-14 illustrate additional embodiments of applying energy in the form of heat to recruit and desensitize pain-associated thermoreceptors. One skilled in the art will recognize that the principles discussed above with respect to FIGS. 8 and 9 can also be applied to the following embodiments.

FIG. 10 is a graph of energy applied versus time illustrating the energy applied to the system and the resultant skin temperature of a patient in accordance with embodiments of the present technology. In FIG. 10, bars 1001 indicate how long and how much energy is applied via the stimulus pods 110, and lines 1002 indicate estimated skin temperature for each profile. The heat applied is measured on an arbitrary scale on the left, and the skin temperature is indicated on an arbitrary scale on the right.

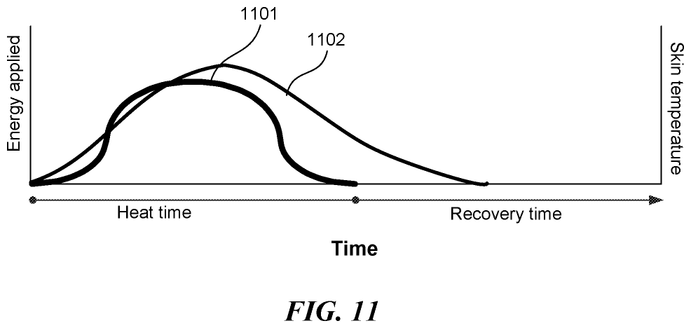

FIG. 11 is a graph of energy applied versus time illustrating a sine wave pattern 1101 of energy applied and the resultant skin temperature 1102 of a patient in accordance with an embodiment of the present technology. In other embodiments, the pattern of applied energy can be a square, crescendo, de-crescendo, intermittent, or any other conceivable pattern. Thus, there are at least five variables that can be adjusted to ensure optimal analgesia; duration of ramp-up time, duration of heating “heat time,” “recovery times” between heat times, intensity of heating, and pattern of heating (sine wave, square wave, saw tooth, etc.).

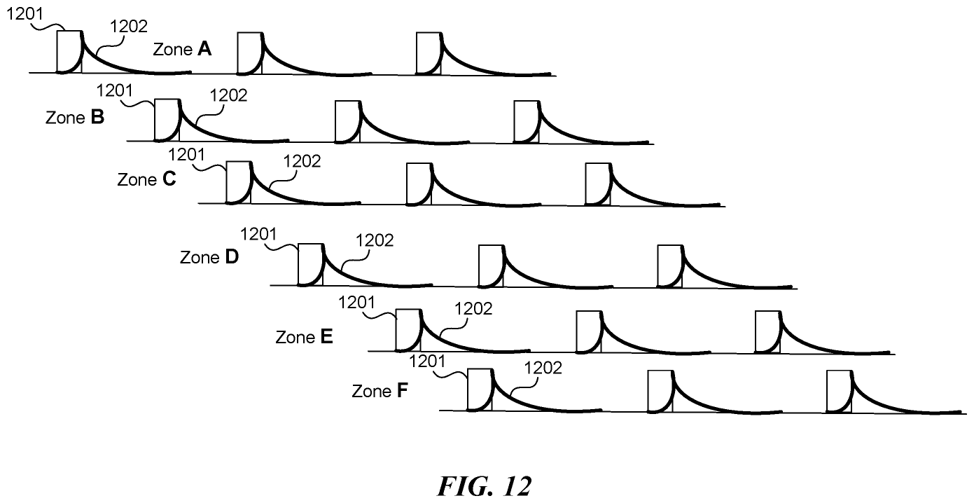

FIG. 12 illustrates energy applied to exemplary thermal zones A, B, C, D, E and the resultant skin temperature of a patient in accordance with embodiments of the present technology. Bars 1201 indicate how long and how much energy is applied, and lines 1202 indicate estimated skin temperature. In some embodiments, the thermal zones A-E can correspond to zones under or proximate to different ones of the stimulus pods 110. In the illustrated embodiment, the skin temperature in the thermal zones A-E has a cascading pattern. In particular, heat is applied to each zone in a sequential pattern. That is, as energy is applied to zone A, zone B rests, then zone B heats while zone A rests, and then zone C heats while zone D rests, and so forth. This has the effect of a wave of heat being passed from zone A to zone E and back again. The principle of moving heat zones can be applied vertically, horizontally, or both to achieve a checkerboard effect or any other conceivable pattern. In other embodiments, the system can deliver any conceivable pattern. For example, heat can be applied in a non-uniform manner. Similarly, by taking advantage of individually controllable heat regions or heat zones (e.g., corresponding to different ones of the stimulus pods 110), the heat can be applied sequentially or in any other imaginable pattern. Sequential heating of individual heat regions as drawn in can enable an entirely different therapeutic sensation to be achieved as compared with heating them all at the same time.

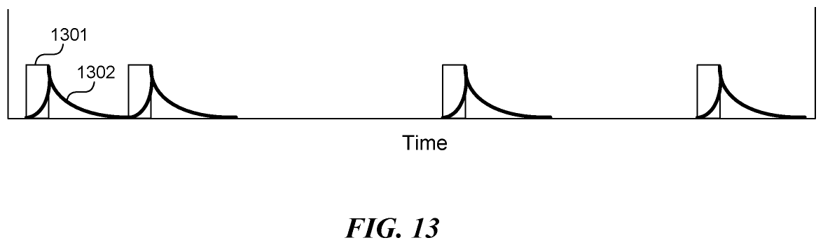

FIG. 13 illustrates an on-demand pattern of variable heat cycles over time as requested by a subject in accordance with an embodiment of the present technology. The patient can press an actuator on the stimulus pod 110, such as a lever, a switch, a pressure sensor, or any other activation device as is known in the art, to demand heat on an as-needed basis. Bars 1301 indicate how long and how much energy is applied, and lines 1302 indicate estimated skin temperature. FIG. 13 shows, on an arbitrary time base, the patient demanding analgesia four times. The pattern of heat delivered by the system could be constant or preprogrammed onto a control unit.

FIG. 14 is a flow diagram illustrating a method 1400 of heat cycling in accordance with embodiments of the present technology. At block 1401, the method 1400 configures the temperature of the heating device to a soak temperature. At block 1402, the method 1400 ramps the temperature of the heating device to a peak temperature and holds the peak temperature for a hold phase (e.g., hold phase 804 as described in FIG. 8). At block 1403, the method 1400 releases the temperature of the heating device to the soak temperature and maintains the soak temperature for a soak phase (e.g., soak phase 808 as described in FIG. 8). The method 1400 can cycle back to block 1402 until the method 1400 ends. The method 1400 can end when a number of cycles is completed and/or a user stops the method 1400. For example, the number of cycles can be one, two, three, five, ten, fifty, hundreds, thousands, or any suitable number of cycles.

The heating cycles described herein have several advantages over previous heat-based therapies. One benefit of certain embodiments of the present technology is the ability to minimize the total amount of heat and/or energy applied to the skin. As previously discussed, pulsing high levels of heat and/or energy with a ramp-up phase of about 4 seconds or less results in less total heat and/or total energy being applied to the skin but is nonetheless effective at recruiting thermoreceptors. For example, a short ramp-up phase (e.g., about 4 seconds or less) recruits more thermoreceptors than a long ramp-up phase. Thus, less total energy must be applied by relying in part on the rapid change of energy to recruit receptors, rather than relying on a total peak energy or temperature. Moreover, by pulsing energy, the benefit of this rapid change of energy may be repeatedly captured through repetitive ramp-up, hold, ramp-down, and soak cycles.

Another advantage of pulsing heat and/or energy is that less total heat and/or energy is applied to the skin through repeated heating cycles, since, during the soak cycles, the heat and/or energy applied to the skin can be minimal. This enables the hold phase of the heat cycle to have higher temperatures than traditional heating therapies, thereby recruiting more thermoreceptors. Accordingly, the heating cycles described herein enable maximal recruitment of thermoreceptors (and therapeutic efficacy) while the tissue remains at a temperature and/or thermal flux below a dangerous level.

Yet another advantage of the variable heat cycles is reduced power demand and consumption during the ramp-down or release phase when the thermal device does not draw power from the power supply or draws reduced power from the power supply. Reduced power consumption results in a more efficient device with a longer life cycle and provides cost savings.

Yet another advantage of the present technology is that the heating devices can be portable and can be conveniently worn by the subject such that pain relief is available as needed. Moreover, in some embodiments, the user may selectively control certain parameters of the heating cycles via a control station such as a cell phone or other device capable of communicating with the stimulus producing device. For example, the user may be able to select, via a touch-screen display or other interactive portion of the control station (e.g., buttons, switches, etc.) a duration and/or pulse frequency for the heat cycling. The user may also select a baseline temperature to maintain during the soak phase and/or a maximum temperature not to surpass during the hold phase. The user can further select the number of heating cycles and/or total duration to apply the heating cycles.

According to aspects of the present technology, the heating devices and heating cycles described herein are designed to relieve pain and/or assist with healing in a variety of medical conditions such as low, mid, or upper back pain, muscular pain, dysmenorrhea, headaches, fibromyalgia, post-herpetic neuralgia, nerve injuries and neuropathies, injuries to extremities, and sprains and strains. The present technology may further be used in conjunction with other therapies known in the art, such as TENS. When combined with other therapies, the present technology may increase the efficacy of these other therapies.

### IV. C NERVE FIBERS, WARM THERMORECEPTORS, AND HEDONIC RESPONSE

The present technology includes systems and methods for applying a stimulus configured to activate C-fibers and warm thermoreceptors of a subject, thus activating the hedonic response and providing relief and reduced pain associated with the hedonic response. For example, the stimulus may be heat, and the stimulus delivery systems described herein can be used to apply the heat to a subject. In another example, other heat sources beyond those explicitly described herein may be used to apply the therapeutic stimuli to the subject. Applying a stimulus in the form of heat can reduce a wide variety of pain and/or treat a variety of ailments associated with the hedonic response. At least a portion of this stimulation-induced pain relief may result from the activation and/or desensitization of warm thermoreceptors.

1. Hedonic Response

To better appreciate the benefits of the present technology, it is helpful to understand the body's hedonic response system and the body's reaction to heat, particularly the hedonic component of temperature sensation. The hedonic response describes an emotional state that can include feelings of calm, relaxation, affection, comfort, soothing, reduced anxiety, and pleasantness. The hedonic response can be associated with reduction in depression, insomnia, anxiety, and stress. Studies using functional magnetic resonance imaging (fMRI) of the brain have shown the hedonic response to be linked with the activation of areas of the brain, including the insular cortex region, anterior cingulate cortex, inferior parietal lobe, caudate nucleus, and frontal regions.

The hedonic response can be stimulated by a number of factors including the presentation of warmth. The hedonic component of temperature sensation includes thermal comfort and discomfort. For example, when warmth is applied locally to the subject and the subject reports feelings of pleasantness, fMRI showed activation of the anterior cingulate cortex, inferior parietal lobe, caudate nucleus, and frontal regions. The sensation of warmth is mediated in humans primarily by C-fibers, which are activated by heat and are involved in the transmission of the feelings of warmth and heat. The hedonic response can be stimulated by the activation of populations of C-fibers and of tactile C-fibers independently from the heat-activated populations of C-fibers.

2. C Nerve Fibers

C-fibers are one class of nerve fibers in the central nervous system (CNS) and peripheral nervous system (PNS). C-fibers are a type of sensory fiber in the peripheral nervous system. Other classes of nerve fibers of the peripheral sensory nervous system include A-beta (Aβ) nerve fibers and A-delta (Aδ) nerve fibers. In general, Aβ nerve fibers (Aβ-fibers) are large in diameter (6-12 μm) and exhibit conduction velocities in the 33-75 m/s range. Aβ-fibers can be stimulated by mechanical movement, touch, and stretch, along with electrical stimulation such as transcutaneous electrical nerve stimulation (TENS). Aδ nerve fibers (A-fibers) are thinner (1-5 μm in diameter) and slower conducting (3-30 m/s) compared to Aβ-fibers. Aδ-fibers can be stimulated by hot and cold temperatures, mechanical factors, and chemicals. Aδ-fibers transmit “fast pain,” such as pain associated with touching a hot stove. C-fibers are the thinnest (0.2-1.5 μm) and slowest conducting (0.5-2 m/s) amongst the other fibers types of the peripheral sensory nervous system. C-fibers are involved in the transmission of warmth, heat, light touch, and pain and can provide a graded response. For example, when exposed to increasing temperatures, C-fibers mediate a sensation of warmth. However, above a certain threshold temperature, approximately 47° C., C-fibers convey a sense of pain and burning. The threshold temperature between stimulating a sensation of thermal comfort and thermal discomfort (e.g., pain) is dependent on the area of the body where heat is applied, the thickness and characteristics of the skin, and the rate of temperature rise when applying heat.

3. TRPV1

The transient receptor potential cation channel subfamily V member 1 (TRPV1 receptor), also known as the capsaicin receptor and the vanilloid receptor 1, is found in C-fibers and plays a role in thermal sensation of warmth and pain. TRPV1 receptors are expressed on Aδ-fibers and C-fibers. Activation of TRPV1 receptors leads to the activation of sensory nerves, such as Aδ-fibers and C-fibers, resulting in the transmission of a pulse leading to the CNS. TRPV1 receptors are also expressed in the CNS and in non-neuronal tissues such as gut and cardiac structures. TRPV1 receptors respond to noxious heat, with an activation threshold temperature of approximately 42-43° C., and to the chemical capsaicin, a vanilloid derived from chili peppers that elicits a burning sensation.

Temperatures to activate C-fibers via TRPV1 receptors include approximately 42-43° C. Temperatures between 38° C. to 49° C. applied at the surface of the skin can stimulate C-fibers. The exact temperature to activate the C-fibers depends on where on the body the temperature is measured, as TRPV1 receptors lie under the skin surface in the dermis and epidermis which leads to variability between individuals. Stimulation of C-fibers also depends on the speed of temperature rise and the baseline tissue temperature. Thermal stimulation applied to the surface of the skin can provide localized and limited activation of TRPV1 receptors, as opposed to systemic activation of TRPV1 receptors. The variability of the temperature threshold for activating TRPV1 receptors depends on tissue factors such as tissue thickness and blood blow. Temperatures too low will not activate TRPV1 receptors, and temperatures too high will be interpreted by the body as noxious pain.

### V. SELECTED EMBODIMENTS OF METHODS OF APPLYING STIMULI TO ACTIVATE THE HEDONIC RESPONSE

The present technology includes methods of applying stimuli to activate the hedonic response and reduce the sensation of pain. For example, certain methods described herein activate the C, Aδ, and Aβ nerve fibers to promote and activate the hedonic response. In some embodiments, the present technology is configured to activate the warm thermoreceptors, such as TRPV1. Certain methods described herein relieve hedonic-related pain, providing feelings of calm, relaxation, affection, comfort, soothing, reduced anxiety, and/or pleasantness. In some embodiments, the present technology promotes the hedonic response associated with a reduction in depression, insomnia, anxiety, and/or stress.

In some embodiments, TRPV1 receptors and C-, Aδ-, and Aβ-fibers are targeted. In some other embodiments, only TRPV1 receptors and C-fibers are targeted. In other embodiments, only C-fibers, only Aδ-fibers, or Aβ-fibers are targeted. The present technology and methods may be advantageous over existing thermal therapies because the present technology may target thermoreceptors and nerve fibers that promote and activate the hedonic response.

The present technology includes applying a stimulus to the skin of a subject. A number of different stimuli may be utilized to activate the hedonic response. For example, thermal, vibratory, mechanical, pressure, electrical, and/or ultrasound energy may be applied to the skin at various areas of the body as stimuli for activating the hedonic response. In some embodiments, thermal stimulation can be combined with electrical, vibrational, mechanical, and/or ultrasound stimulation to activate the hedonic response. In other embodiments, only thermal, only electrical, only vibrational, only mechanical, or only ultrasound stimulation is used to activate the hedonic response. The stimuli can induce a change in a mental and/or emotional state of a person or animal through the application of stimuli to the skin.

In some embodiments, thermal energy (e.g., heat) may be applied at any temperature greater than typical skin temperatures, which is approximately 33.5 to 36.9° C. In the illustrated example, heat may be applied at about 35° C., 36° C., 37° C., 38° C., 39° C., 40° C., 41° C., 42° C., 43° C., 44° C., 45° C., 46° C., 47° C., 48° C., and/or 49° C. The hedonic response may be activated by such temperatures. A higher temperature of heat may be applied to the skin to activate a greater hedonic response. For example, temperatures greater than 40° C. but below temperatures that cause tissue damage can be applied to an area of skin to promote a greater hedonic response. Various forms of heat may be applied to an area of the skin to stimulate the hedonic response. For example, conductive heat and/or radiant heat (e.g., infrared heat) may be applied to skin.

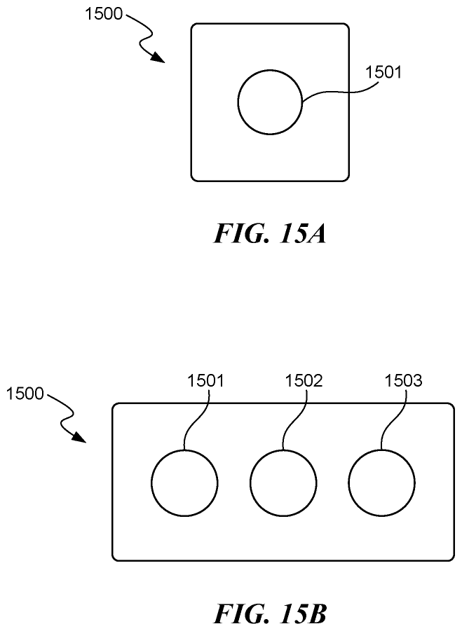

FIGS. 15A and 15B illustrate a hedonic device 1500 for activating the hedonic response configured in accordance with embodiments of the present technology. The hedonic device 1500 can apply different stimuli to a subject's body, including heat, vibration, electricity, pressure, light, ultrasound, and/or other different stimuli. The hedonic device 1500 may apply stimuli to a surface area that is at least 0.5 square inches (3.23 cm²). In FIG. 15A, the hedonic device 1500 includes a heating device 1501. The heating device 1501 is configured to apply continuous and/or intermittent heat. The heating device 1501 can be generally similar to or the same as stimulus pod 110. For example, the heating device 1501 can be configured to pulse and cycle heat as described in detail above with reference to FIGS. 1-14. For example, the temperature during a hold phase of heat cycling may be greater than about 40° C. and less than a temperature that can cause tissue damage. A hold phase and a peak-time hold phase may be within a range between 0.5 seconds to 120 minutes. An advantage of using stimulus pods 110 configured to pulse and cycle heat is that more thermal energy (e.g., higher temperatures) can be applied to induce a larger hedonic response.

FIG. 15B illustrates another embodiment of the hedonic device 1500. The hedonic device 1500 can include the heating device 1501, a vibrating device 1502, and an electrical device 1503. The vibrating device 1502 can be generally similar to or the same as the stimulus pod 110, and is configured to apply continuous and/or intermittent vibrations. The electrical device 1503 can be generally similar to or the same as the stimulus pod 110, and is configured to apply continuous and/or intermittent electricity. Stimuli, such as heat, heat pulses, vibration, and electricity, can be applied to the skin in patterns to stimulate the nervous system and create a sensation akin to stroking, rubbing, or moving touch. The stimuli may guide breathing, heart rate, blood pressure, and/or brain wave activity.

While the hedonic device 1500 is illustrated to include one heating device 1501 (FIG. 15A) or one heating device 1501, one vibrating device 1502, and one electrical device 1503 (FIG. 15B), the hedonic device 1500 can include any suitable number of heating devices, vibrating devices, electrical devices, and/or devices that apply other different stimuli. For examples, the hedonic device 1500 can include only one or more heating devices, only one or more vibrating devices, only one or more electrical devices, only one or more mechanical devices, only one or more light-emitting devices, or only one or more ultrasound devices. In another example, the hedonic device 1500 can include a combination of one or more heating devices, vibrating devices, electrical devices, mechanical devices, light-emitting devices, and ultrasound devices. In this illustrated example, the hedonic device 1500 can include: a) the heating device and the vibrating device, b) the heating device, the vibrating device, and the electrical device, or c) the heating device and the mechanical device. When the heating device is combined with other devices, the present technology may increase the efficacy of these other devices during the promotion of the hedonic response.

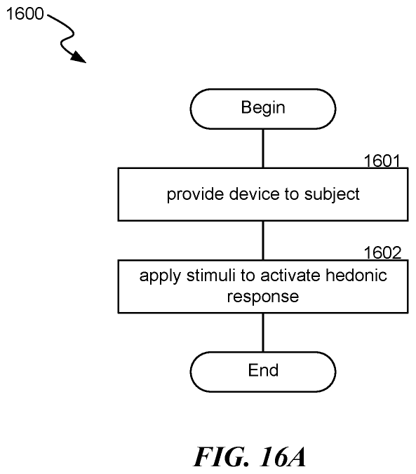

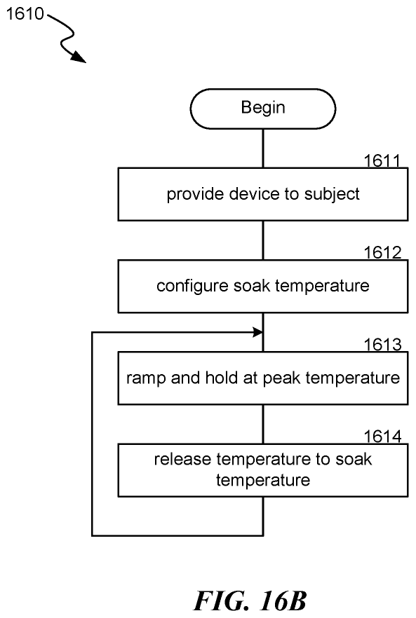

FIG. 16A is flow diagram of a method 1600 for activating the hedonic response of a subject in accordance with embodiments of the present technology. At block 1601, the method 1600 provides a hedonic device to a subject for activating the hedonic response. At block 1602, the method 1600 includes configuring the hedonic device to apply stimuli to the subject to activate the subject's hedonic response. The hedonic device is applied to a surface area of the skin of the subject. The stimuli can be thermal, vibratory, mechanical, pressure, electrical, and/or ultrasound energy.

FIG. 16B is a flow diagram of a method 1610 for activating the hedonic response of a subject in accordance with embodiments of the present technology. The method 1610 is generally similar to the method 1400 of applying heat intermittently as described in reference to FIG. 14. At block 1611, the method 1610 includes providing a hedonic device to a surface area of the skin of a subject for activating the hedonic response. At block 1612, the method 1610 includes setting the temperature of the hedonic device to a soak temperature. At block 1613, the method 1610 includes ramping the temperature of the hedonic device to a peak temperature for the duration of a hold phase. At block 1613, the method 1610 includes releasing the temperature of the hedonic device to the soak temperature and maintains the soak temperature for the duration of a soak phase. The method 1610 can cycle back to block 1613 until the method 1610 ends. The method 1610 can end when a number of cycles is completed and/or a user stops the method 1610. For example, the number of cycles can be one, two, three, five, ten, fifty, hundreds, thousands, or any suitable number of cycles.

The combination of the continuous low-level heating and intermittent high-level heating at discrete, focused regions provides several advantages over conventional heating systems. The augmentation of the continuous heating (or cooling), for example, provides enhanced hedonic responses. The illustrated configuration achieves enhanced promotion of the hedonic response by providing a strong stimulation of the thermoreceptors in the skin and nerve fibers of the body by rapidly changing temperatures. Both the rapid change in temperature (e.g., the rapid increase in temperature during the intermittent burst) and the ability to stimulate at higher temperatures (e.g., above about 40° C.) recruits more thermoreceptors, including TRPV1, and greater stimulation of nerve fibers, including C-, Aδ-, and Aβ-fibers. Accordingly, the intermittent focused bursts of heat, combined with the constant heat, provide for better receptor recruitment and stimulation, thereby leading to increased activation of hedonic responses.

In some embodiments, activation of the hedonic response may supplement other methods and/or devices, such as methods and/or devices for meditation. Meditation can include self-relaxation and self-management techniques, along with techniques that promote relaxation, mindfulness, calmness, and guided imagery. For example, the heated pulse cycling may be synced with breathing, heart rate, blood pressure, and/or brain wave activity. In a further example, the heated pulse cycling may guide breathing, heart rate, blood pressure, and/or brain wave activity. In a further example, the heated pulse cycling may be used to reinforce or support relaxation exercises such as breathing.

An advantage of the present technology is that the devices can be portable and can be conveniently worn by the subject such that hedonic response promotion is available as needed. Moreover, in some embodiments, the user may selectively control certain parameters of the heating cycles via a control station such as a cell phone or other device capable of communicating with the stimulus producing device. For example, the user may be able to select, via a touch-screen display or other interactive portion of the control station (e.g., buttons, switches, etc.) a duration and/or pulse frequency for the heat cycling. The user may also select a baseline temperature to maintain during the soak phase and/or a maximum temperature not to surpass during the hold phase. The user can further select the number of heating cycles and/or total duration to apply the heating cycles.

According to aspects of the present technology, the devices and methods described herein are designed to activate the hedonic response and relieve pain associated with the hedonic response. The present technology can assist with providing feelings of calm, relaxation, affection, comfort, soothing, reduced anxiety, and/or pleasantness and reduce depression, insomnia, anxiety, and/or stress.

### VI. THERMORECEPTORS AND APPETITE SUPPRESSION

The present technology includes systems and methods for applying a stimulus configured to activate thermoreceptors of a subject and suppress appetite. For example, the stimulus may be heat, and the stimulus delivery systems described herein can be used to apply the heat to a subject. In another example, other heat sources beyond those explicitly described herein may be used to apply the therapeutic stimuli to the subject. Applying a stimulus in the form of heat, vibration, electricity, and/or pressure can suppress appetite. At least a portion of this stimulation-induced appetite suppression may result from the activation and/or desensitization of thermoreceptors.

To better appreciate the benefits of the present technology, it is helpful to understand the role of thermoreceptors, particularly the TRPV1 receptor, in appetite regulation. TRPV1 receptors are involved in energy homeostasis. Energy homeostasis is important in maintaining a healthy body weight and losing weight by expending more energy than energy intake. The activation TRPV1 receptors on peripheral nerves is associated with central stimulation that reinforces the feeling of satiety, thereby reducing appetite. The activation of TRPV1 receptors, such as by heat, may influence appetite by controlling hormone levels associated with appetite.

### VII. SELECTED EMBODIMENTS OF METHODS OF APPLYING STIMULI TO SUPPRESS APPETITE

The present technology includes methods of applying stimuli to suppress appetite. For example, certain methods described herein activate the C-fibers to suppress appetite. In some embodiments, the present technology is configured to activate the warm thermoreceptors, such as TRPV1. In some embodiments, TRPV1 receptors and C-, Aδ-, and Aβ-fibers are targeted. In some other embodiments, only TRPV1 receptors and C-fibers are targeted. In other embodiments, only C-fibers, only Aδ-fibers, or only Aβ-fibers are targeted. The present technology and methods may be advantageous over existing therapies because the present technology may target thermoreceptors and nerve fibers that promote the feeling of satiety and suppress appetite.

The present technology includes applying a stimulus to the skin of a subject. A number of different stimuli may be utilized to suppress appetite. For example, thermal, vibratory, mechanical, pressure, electrical, and/or ultrasound energy may be applied to the skin at various areas of the body as stimuli for suppressing appetite. In some embodiments, thermal stimulation can be combined with electrical, vibrational, mechanical, and/or ultrasound stimulation to suppress appetite. In other embodiments, only thermal, only electrical, only vibrational, only mechanical, or only ultrasound stimulation is used to promote appetite suppression.

In some embodiments, thermal energy (e.g., heat) may be applied at any temperature greater than typical skin temperatures, which is approximately 33.5 to 36.9° C. In the illustrated example, heat may be applied at about 35° C., 36° C., 37° C., 38° C., 39° C., 40° C., 41° C., 42° C., 43° C., 44° C., 45° C., 46° C., 47° C., 48° C., and/or 49° C. Feelings of satiety and appetite suppression may be activated by such temperatures. A higher temperature of heat may be applied to the skin to achieve greater suppression of appetite. For example, temperatures greater than 40° C. but below temperatures that cause tissue damage can be applied to an area of skin to achieve greater suppression of appetite. Various forms of heat may be applied to an area of the skin to achieve suppression of appetite. For example, conductive heat and/or radiant heat (e.g., infrared heat) may be applied to skin.

The present technology includes applying stimuli to a subject and monitoring physiological parameters using methods and/or devices as described in FIGS. 1-17 for suppressing appetite. For example, pulsed heating from the stimulus pod 110 may be applied to a surface area of the skin of a subject to suppress appetite. In another example, pulsed heating, vibrational stimuli, and electrical stimuli from the device 1500 may be applied to a surface area of the skin of a subject to suppress appetite.

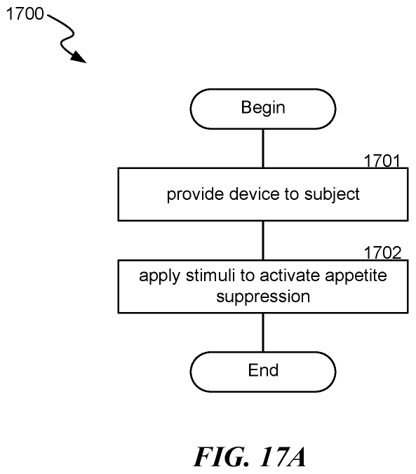

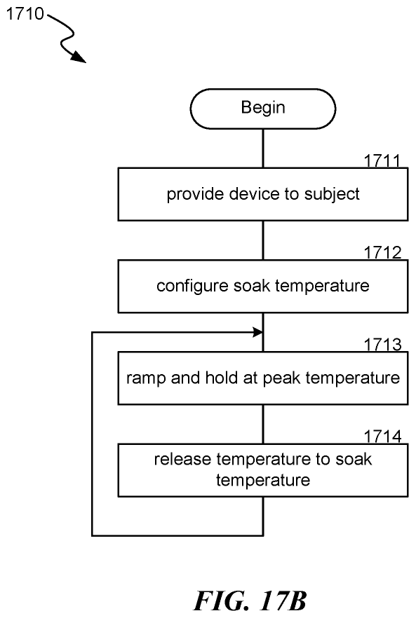

FIG. 17A is a flow diagram of a method 1700 for suppressing the appetite of a subject in accordance with embodiments of the present technology. At block 1701, the method 1700 provides an appetite suppressant device to a subject for suppressing appetite. At block 1702, the method 1700 includes configuring the hedonic device to apply stimuli to the subject to activate the subject's hedonic response. The hedonic device is applied to a surface area of the skin of the subject. The stimuli can be thermal, vibratory, mechanical, pressure, electrical, and/or ultrasound energy.

FIG. 17B is a flow diagram of a method 1710 for suppressing the appetite of a subject in accordance with embodiments of the present technology. The method 1710 is generally similar to the method 1400 of applying heat intermittently as described in reference to FIG. 14. At block 1711, the method 1710 includes providing an appetite suppressant device generally similar to or the same as stimulus pod 110 to a subject. At block 1712, the method 1710 includes configuring the temperature of the hedonic device to a soak temperature. At block 1713, the method 1710 includes ramping the temperature of the appetite suppressant device to a peak temperature for the duration of a hold phase. At block 1714, the method 1710 includes releasing the temperature of the appetite suppressant device to the soak temperature and maintains the soak temperature for the duration of a soak phase. The method 1710 can cycle back to block 1713 until the method 1710 ends. The method 1710 can end when a number of cycles is completed and/or a user stops the method 1710. For example, the number of cycles can be one, two, three, five, ten, fifty, hundreds, thousands, or any suitable number of cycles.

### VIII. SELECTED EMBODIMENTS OF APPLYING STIMULI AND MONITORING PHYSIOLOGICAL PARAMETERS

The present technology includes combining methods of applying stimuli with monitoring physiological parameters of a subject. For example, a device applying stimuli to the subject (e.g., one or more of the stimulus pods 110 and/or the hedonic device 1500) can be combined with one or more monitoring devices that measure physiological parameters of the subject. Physiological parameters can include blood flow, oxygenation, temperature, and a change in temperature over time through an area of the user's skin and/or muscle, muscle tension and activity, and physical movement. The physiological parameters can be recorded and/or transmitted as feedback data to a control device and/or an interface that the subject and/or a third-party user (e.g., a physician and physical therapist) interacts with. The feedback data provided to the subject and/or third-party user can be used to guide therapy, evaluate progress (e.g., level of pain relief), and/or compare to other patients' data.

In some embodiments, the feedback data may also be used to guide treatment of the patient. Treatment can include applying stimuli to the subject. A user can be the subject and/or a third-party user (e.g., a healthcare provider). The user can view the feedback data and/or input preferences to configuring a stimulus device based on the feedback data presented to the user. The user preferences can include adjustments to physiological parameters (e.g., blood flow, oxygenation level, and skin temperature), duration of applying stimuli (e.g., 10 seconds, 10 minutes, and 1 hour), type of stimuli (e.g., heat, vibration, electricity, pressure, and light), and other suitable parameters for adjustment. For example, the user preferences can include increase blood flow by 20%, increase oxygenation by 20%, and decrease skin temperature by 20% for ten minutes. In another example, the user preferences can include apply heat intermittently for 10 minutes. A program can calculate the required settings for configuring the stimulus device in order to achieve the user preferences.

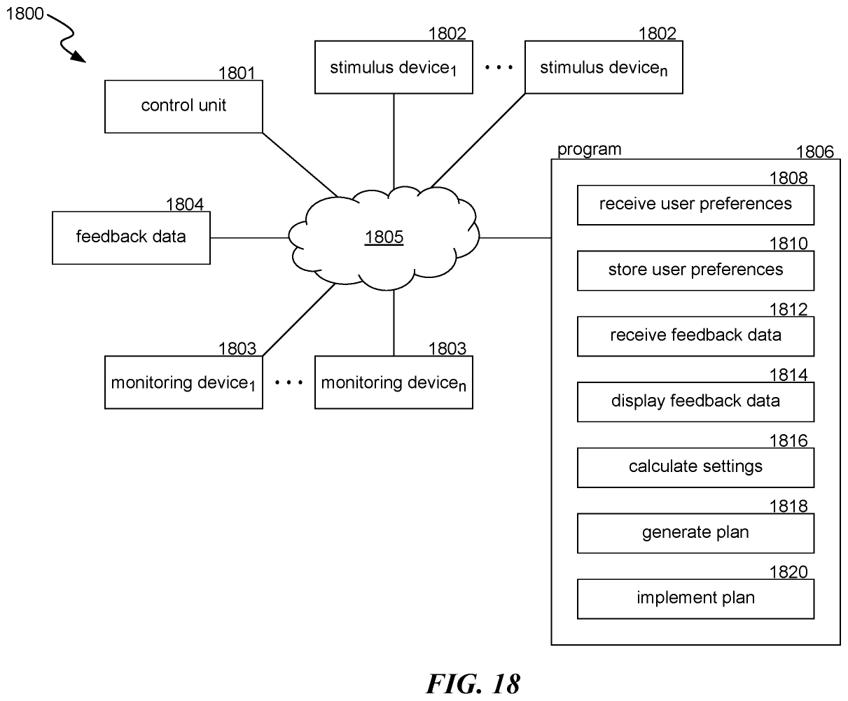

FIG. 18 illustrates a system 1800 for delivering a stimulus and monitoring physiological parameters of a subject configured in accordance with embodiments of the present technology. The system 1800 can include a control unit 1801, one or more stimulus devices 1802, one or more monitoring devices 1803, feedback data 1804, facility 1805, and a program 1806. The facility 1805 can provide systems and methods for controlling the control unit 1801, stimulus devices 1802, monitoring devices 1803, feedback data 1804, and program 1806. The program 1806 can include component 1808 configured to receive user preferences, component 1810 configured to store user preferences, component 1812 configured to receive feedback data from monitoring devices 1803, component 1814 configured to display feedback data, component 1816 configured to calculate settings for stimulus devices 1802 based on the user preferences, component 1818 configured to generate a plan based on the calculated settings, and component 1820 configured to implement the generated plan. The user can input preferences via the control unit 1801 and/or the stimulus device 1802. The plan can include settings to configure the stimulus devices 1802 and/or the monitoring devices 1803. For example, the plan can include intermittently applying a thermal stimulus to a surface area of the skin at 45° C. for 30 minutes. In another example, the plan can additionally include monitoring temperature of the surface area of the skin at intervals of 2 minutes and applying a thermal stimulus such that the monitored temperature is about 45° C.

The program 1806 can be embodied in a non-transitory computer-readable storage medium that stores instructions that when executed by a processor, carry out the functions attributed to the program 1806. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The present technology can be embodied in a special purpose computer or data processor that is specifically programmed, configured or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

In some embodiments, the stimulus device 1802 can provide thermal, vibratory, mechanical, electrical, light, and/or ultrasound energy. The monitoring device 1803 can monitor physiological parameters including, but not limited to, heart rate, respiratory rate, blood pressure, muscle tension, electrical activity of the brain, blood flow, oxygenation, body temperature, skin temperature, perspiration, limbic movement, and/or hormone level. The control unit 1801 can be a desktop or laptop computer, a smartphone, a tablet, or other device. The control unit 1801 can communicate with the stimulus device 1802 and/or monitoring device 1803 through any accepted wireless or wired protocol, including radio frequency (RF), infrared light, laser light, visible light, acoustic energy, BLUETOOTH, WIFI, or other communication systems. Additionally, the signals can be sent and received through the subject's skin.

In some embodiments, the stimulus device 1802 can be generally similar to or the same as stimulus pod 110. The monitoring device 1803 can be generally similar to or the same as a pulse oximeter, heart rate monitor, devices used for electroencephalography (EEG), devices used for electromyography (EMG), goniometer-like devices, thermometer, and other devices and/or sensors for monitoring physiological parameters.

In some embodiments, the stimulus device 1802 and the monitoring device 1803 can be standalone devices located proximate to each other. For example, the stimulus device 1802 can be applied to a surface area of the skin and configured to apply heat to the surface area of the skin, and the monitoring device 1803 can be applied in the same or proximate area of the surface area of the skin configured to monitor skin temperature, blood flow, and/or oxygenation. In other embodiments, the stimulus device 1802 and the monitoring device 1803 can be integrated into an individual unit.

In some embodiments, the stimulus device 1802 and the monitoring device 1803 can be standalone devices located apart from each other. For example, the stimulus device 1802 can be applied to a surface area of the skin on the neck of a subject and configured to apply heat to the surface area of the skin for activating the hedonic response, and the monitoring device 1803 can be applied to an area of the scalp of the subject and configured to monitor electrical activity of the brain to monitor the hedonic response. In a further example, a second monitoring device 1803 can be applied to an area of the chest to monitor respiratory rates, and a third monitoring device 1803 can be applied to an area of the wrist to monitor heart rate.

In some embodiments, the control unit 1801, the stimulus device 1802, and the monitoring device 1803 can be standalone devices. In one embodiment, the stimulus device 1802 and the monitoring device 1803 can be located proximate to each other (i.e., co-localized). For example, the stimulus device 1802 can include the stimulus pod 110 and can be located next to the monitoring device 1803 that is a thermometer. In another embodiment, the stimulus device 1802 and the monitoring device 1803 are located apart from each other. For example, the stimulus device 1802 can include the stimulus pod 110 that is attached to the neck, and the monitoring device 1803 can be a pulse oximeter that is attached to a finger.

In some embodiments, the stimuli provided by the stimulus device 1803 can be synchronized with physiological parameters, such as the subject's brain waves, heart rate, blood pressure, and/or respiratory variability. In one embodiment, applying stimuli to a subject can be synchronized with the subject's brain waves. One or more monitoring devices 1803 can monitor and measure the electrical activity of the brain. For example, the monitoring device 1803 can be an EEG device for recording brain activity and a quantitative EEG (qEEG) device for analyzing the recordings. The analyzed output of brain activity can be brain waves (neural oscillations). The brain waves can be used as feedback data for calculating settings for the stimulus device 1802. The stimulus pod can be generally similar to or the same as stimulus pod 110. The plan generated from the program 1806 can include parameters for pulsing and cycling heat as described in FIGS. 1-14. For example, the parameters can include a soak temperature, hold temperature, soak time, hold time, and number of cycles. The parameters can be selected to synchronize with the frequency of the brain waves. In some embodiments, the parameters for pulsing and cycling heat can be synchronized with alpha waves (neural oscillations in the frequency range of 8-12 Hz) to promote the hedonic response. Alpha waves are associated with feelings invoked by the hedonic response, including relaxed, calm, and lucid.

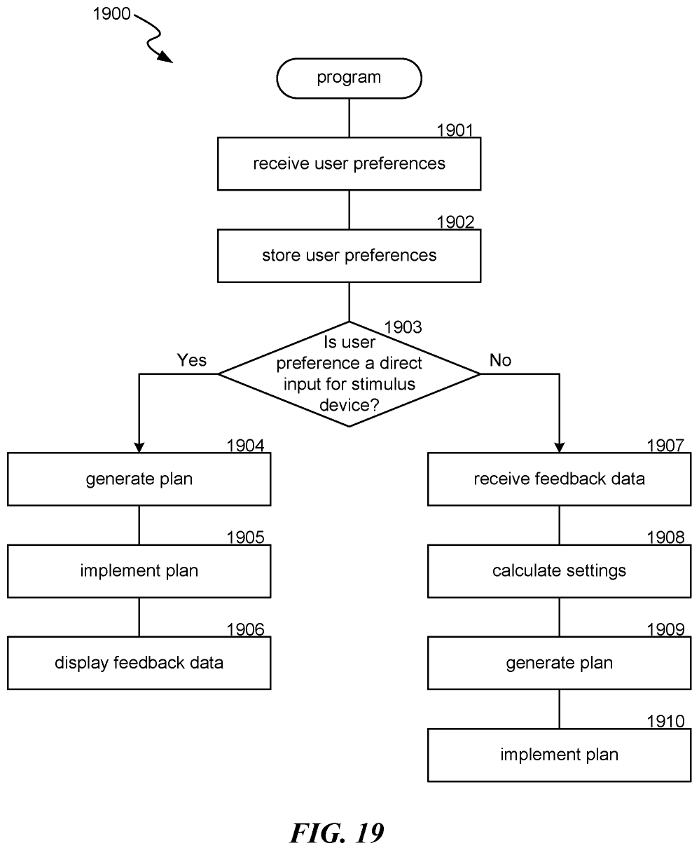

FIG. 19 is a flow diagram illustrating the processing of a program component in accordance with some embodiments of the present technology. The program component can be invoked by a user to generate and implement a plan for a stimulus device and/or monitoring device to achieve user preferences/goals. At block 1901, the component receives user preferences. At block 1902, the component stores the user preferences. At decision block 1903, the component determines if the user preference is a direct input for a stimulus device. Direct inputs for the stimulus device can include parameters for adjusting one or more stimulus devices. For example, if a stimulus device provides heat, direct inputs can include temperatures (e.g., 45° C.), duration of heat (e.g., 5 minutes), and pattern of heat (e.g., continuous and intermittent). User preferences that are not considered direct inputs can include parameters for physiological adjustments. For example, the user preferences can include increase blood flow by 20%, increase oxygenation by 20%, decrease skin temperature by 20%, and increase range of motion around the elbow. If the user preference is a direct input for the stimulus device, then the component continues at block 1904, else the component continues at block 1907. At block 1904, the component generates a plan. At block 1905, the component implements the plan. The plan can include settings to configure one or more stimulus devices. For example, the plan can include configuring one stimulus device to apply intermittent heat at 45° C. for 5 minutes and/or another stimulus device to apply intermittent vibrations for 5 minutes. At block 1906, the component displays feedback data for the user to view. The user may adjust direct inputs for the stimulus device based on feedback data. For example, the displayed feedback data may include a heart rate of the user, and the user may desire to adjust the settings for the stimulus device such as apply intermittent heat for 10 minutes. At block 1907, the component received feedback data from one or more monitoring devices. At block 1908, the component calculates settings for one or more stimulus devices based on the feedback data. At block 1909, the component generates a plan. At block 1910, the component implements the plan. The plan can include settings to configure the one or more stimulus devices and/or settings to configure the one or more monitoring devices. For example, the plan can include configuring the stimulus device to intermittently apply heat to a surface area of the skin at 45° C. for 30 minutes and configuring the monitoring device to monitor electrical activity in the brain.

Any of the systems, devices, and programs illustrated in FIGS. 18 and 19 provide a way for applying stimuli to the subject and monitoring physiological feedback. These systems, devices, and programs can be combined and/or integrated together with the systems, devices, and methods discussed in reference to FIGS. 1-17B. For example, the stimulus device 1802 can be generally similar to or the same as stimulus pod 110. In another example, user preferences can include relaxation, and the program 1806 can include components for activating the hedonic response to carry out the steps in the method 1600 and/or method 1610. In a further example, user preferences can include appetite suppression, and the program 1806 can include components for suppressing appetite to carry out the steps in the method 1700 and/or 1710.

### IX. THERMAL EFFECTS ON MUSCLES, TENDONS, AND LIGAMENTS

The present technology includes methods of applying stimuli to soft tissues, including muscles, tendons, and ligaments. Certain methods described herein provides relaxation of muscles, tendons, and ligaments for physical movements, such as physical therapy, exercise warm-ups, and stretching. Certain methods described herein relieve pain and/or promote recovery associated with exercise, recreation, and/or physical therapy. For example, it may be used prior to, during, and/or after exercise and/or treatment during physical therapy. In a further example, it may be used to improve performance and/or reduce the risk of injury. In some embodiments, the present technology promotes an increased range of motion around the joints of a subject's body. The joints can include the major and minor axial joints (e.g., knees, shoulders, hips, ankle, elbows, wrists, fingers, toes, and jaw), areas of the back including from the joints contained in the neuraxial structures from the first cervical vertebrate through the last lumbar vertebra, and the sacral joint. In some embodiments, the present technology may augment in-clinic and/or out-of-clinic physical therapy.

To better appreciate the benefits of the present technology, it is helpful to understand the effects of local heating on muscles and ligaments. Local heating of muscles (e.g., applying external heat to an area near the target muscle) may increase muscle blood flow. For example, applying local heat to a calf muscle of a subject can increase the intramuscular temperature and blood flow in the calf. External tissue warming may increase the flexibility of connective tissue, such as muscles tendons and ligaments. Applying heat to the area of the connective tissue prior to or after exercise may result in faster recovery and less injuries in both animals and humans. For example, applying heat to a subject undergoing physical therapy for neck pain can promote a greater range of motion, pain relief, and compliance for exercise. In another example, supplementing heat therapy with a heat wrap to a subject experiencing lower back pain can promote strength and flexibility. In a further example, applying heat as thermal treatment to subjects undergoing physical therapy for knee injuries is associated with a statistically significant reduction in pain and an increase in range of motion, compliance, and strength. Furthermore, applying heat to muscles prior to exercise may reduce the effects of repeated muscle motion and associated fatigue. For example, applying heat to the lower back area before and after performing exercises associated with that area may reduce delayed onset muscle soreness. Applying heat to muscles prior to exercise may also reduce markers of cell damage (e.g., creatine phosphokinase) and post-exercise related pain. Local heating of the skin may increase muscle blood flow and improve oxygen and/or metabolite delivery, thereby leading to a reduction in byproducts of injury and an expedition of recovery and healing of the muscles and/or ligaments.

### X. SELECTED EMBODIMENTS OF METHODS OF APPLYING STIMULI TO IMPROVE RECOVERY ASSOCIATED WITH PHYSICAL MOVEMENTS

The present technology includes applying stimuli to a subject and monitoring physiological parameters of a subject for relieving pain, reducing risk of injury, improving recovery of muscles and ligaments, and increasing a range of motion associated with physical movements. For example, the stimulus may be heat, and the stimulus delivery systems described herein can be used to apply the heat to the subject. In another example, other heat sources beyond those explicitly described herein may be used to apply the therapeutic stimuli to the subject. In a further example, physiological parameters monitored may be muscle blood flow and oxygenation. Applying a stimulus in the form of heat can reduce a wide variety of pain and/or treat a variety of ailments associated with physical activity and exercise.

As described herein, certain methods in accordance with the present technology may utilize the devices, systems, and methods described in FIGS. 1-14, 18, and 19 for applying stimuli and/or monitoring physiological parameters. For example, pulsed heating and/or continuous heating from the stimulus device 1802 (FIG. 18; including features generally similar to the stimulus devices discussed above) may be applied to an area of the skin of a subject around the joints to promote blood flow and recovery from exercise. In another example, pulsed heating and/or continuous heating from the stimulus device 1802 may be applied to an area of the subject's skin proximate to one or more muscles to promote muscle relaxation. In a further example, the system 1800 and/or the method 1900 described in FIGS. 18 and 19, respectively, can include utilizing devices that monitor physiological parameters and/or physical body motions (e.g., goniometer-like devices) in combination with the stimulus device 1802 that emit pulsed heat and/or continuous heat for increasing a range of motion for the subject. These monitoring devices 1803 (FIG. 18) can measure or otherwise receive feedback data, including various body temperatures, brain activity, blood flow, oxygen delivery, muscle tension, motion outcomes, pain outcomes, and/or treatment cycle, which can be transmitted to the user.

In some embodiments, the systems and methods disclosed herein can include a combination of pulsed heating, followed by a period of continuous heat application at steady temperatures. One or more stimulus devices (e.g., the stimulus device 1802 of FIG. 18) can apply pulsed heat and continuous heat to an area of the skin of a human subject (e.g., an area of at least 1.27 cm²), such as around the joints to promote blood flow and recovery from exercise and/or at one or more muscles to promote muscle relaxation. The pulsed heat can be applied for a first period at a first temperature range. For example, the first temperature range can be between 40° C. and 60.5° C. The continuous heat can be applied for a second period at a second temperature. For example, the second temperature can be above 40° C. and less than the maximum temperature of the pulsed heating (e.g., less than 60.5° C.). The treatment protocol (via a controller) can instruct one or more stimulus devices to apply the pulsed heating before the continuous heating period or the continuous heating can occur prior to pulsed heating. In various embodiments, the treatment protocol can cause one or more stimulus devices to alternate between pulsed and continuous heating. In systems and methods that provide combinations of pulsed and continuous heating protocols, the pulsed heat is expected to reduce pain, while subsequent steady heat application is expected to increase muscle temperature and/or blood flow.

During the application of the pulsed and/or continuous heating, one or more monitoring devices (e.g., the monitoring devices 1803 of FIG. 18) can measure one or more physiological parameters, including blood flow, tissue oxygenation, muscle tension, muscle activation, skin temperature, subcutaneous temperature, muscle temperature, and/or skin thermal flux via direct or inferred measurements. The detected physiological parameters can be compared to predetermined physiological parameters indicative of desired outcomes or metrics, such as nerve stimulation, reduction of muscle activity or tension, subdermal tissue heating, and/or pain relief. Accordingly, treatment parameters of the one or more stimulus devices, such as temperature, pulse length, pulse frequency, and continuous heating length, can be defined such that the applied continuous heat and/or the applied pulsed heat is administered in a manner that produces measured physiological parameters (detected via the monitoring device(s)) that correspond with the predefined values associated with the desired outcome. For example, the treatment protocol can synchronize the pulsed or continuous heating to produce measured physiological parameters corresponding to nerve fiber stimulation, thermoreceptor stimulation, reduction of muscle activity and tension, and/or other desired outcomes or metrics.

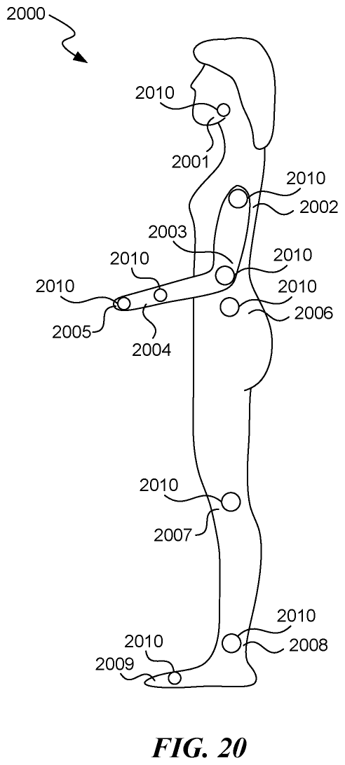

FIG. 20 is a side view of a human form 2000 wearing a plurality of stimulus devices 2010 in accordance with select embodiments of the present technology. The stimulus devices 2010 can generally be similar to or the same as stimulus pods 110, hedonic device 1500, and/or stimulus devices 1802. The stimulus devices 2010 can be configured to provide pulsed heat and/or continuous heat to various joints of the body 2000, including but not limited to, jaw 2001, shoulder 2002, elbow 2003, wrist 2004, finger 2005, hip 2006, knee 2007, ankle 2008, and toe 2009. The stimulus device 2010 can be attached to a wearable garment (not illustrated) as a wearable device to facilitate the use on joints. For example, the wearable garment can include the stimulus device 2010 and configured to be worn around the knee 2007. In a further example, the stimulus device 2010 included in the wearable garment can be connected, via wired or wireless means, to a control unit (not illustrated). A user can control the stimulus device 2010 via the control unit.

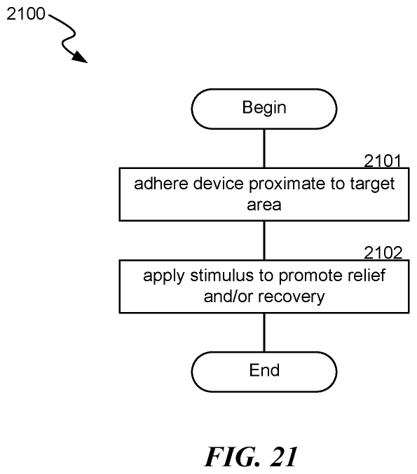

FIG. 21 illustrates a flow diagram of a method 2100 for promoting pain relief and recovery around the joints, muscles, tendons, and/or ligaments of a human body in accordance with embodiments of the present technology. At block 2101, the method 2100 includes applying the stimulus device 2010 proximate to a target area, such as joints, muscles, tendons, and/or ligaments of a subject. At block 2102, the method 2100 configures the device 2010 to apply a stimulus to promote relief and/or recovery in the target area. The stimuli can be thermal, vibratory, mechanical, pressure, electrical, and/or ultrasound energy. In some embodiments, the stimulus is applied as pulsed heat.

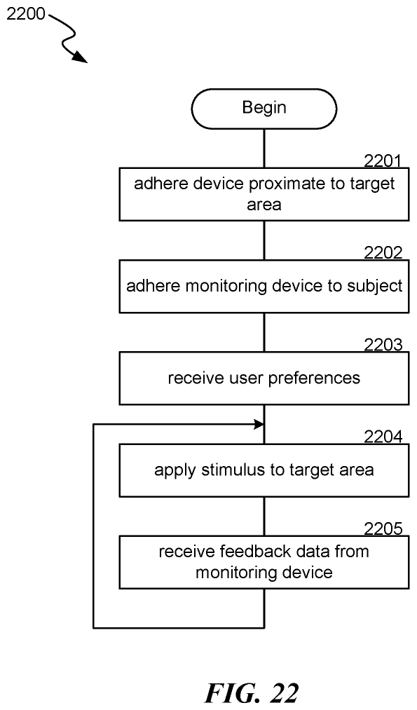

FIG. 22 illustrates a flow diagram of a method 2200 for promoting pain relief and recovery around the joints, muscles, tendons, and/or ligaments of a human body in accordance with embodiments of the present technology. At block 2201, the method 2200 adheres stimulus device 2010 proximate to a target area, such as joints, muscles, tendons, and/or ligaments of a subject. At block 2202, the method 2200 adheres one or more monitoring devices to the subject. For example, a heart rate monitor can be applied to the chest of the subject, a pulse oximeter can be applied to the index finger of the subject, and a thermometer can be applied to armpit of the subject. At block 2203, the method 2200 receives user preferences from a user (e.g., the subject and/or a third-party user). User preferences can include, but are not limited to, increase blood flow to target area, increase oxygenation in target area, and increase range of motion. At block 2204, the method 2200 applies a stimulus to the target area. For example, the stimulus can be pulsed heat. In another example, the stimulus can be continuous heat. At block 2205, the method 2200 receives feedback data from the monitoring devices for the user to view. The method 2200 cycles back to block 2204 until the method 2200 ends. The method 2200 can end when user preferences are met and/or when the user stops the method 2200.

### XI. SELECTED EMBODIMENTS OF A ROTATING STIMULUS DEVICE

The present technology includes methods of applying stimuli to a subject to reduce neural accommodation and/or the chance of injury related to thermal heat. In some embodiments, the present technology is configured to activate individual stimulating elements asynchronously within a group of stimulating elements on a subject. The advantage of asynchronous activation of individual stimulating elements within a group may be reduced neural accommodation and risk of tissue damage associated with prolonged duration of applying the stimulus. Yet another advantage is reduced neural accommodation and/or tissue damage while inducing the subject to feel a prolonged duration of the stimulus in the overall target area. Asynchronous activation of each stimulating element allows for shorter durations of applying continuous stimuli and/or applying the stimuli at higher energies (e.g., higher temperatures, stronger vibrations, and increased pressure). In some embodiments, multiple stimulating elements may be activated simultaneously. An advantage of simultaneous activation of multiple stimulating elements includes achieving a greater level of activation of peripheral receptors (e.g., thermoreceptors) at lower levels of energy (e.g., lower temperatures). For example, the level of activation of peripheral receptors can be generally similar to a level of activation at higher levels of energy (e.g., higher temperatures) using an individual stimulating element. The simultaneous activation of multiple stimulating elements may achieve similar to or better results compared to the activation of a lesser number of stimulating elements that applies higher energy (e.g., higher temperatures). For example, the results include greater user comfort (e.g., greater hedonic response) and/or safety (e.g., decreased risk of skin burn).

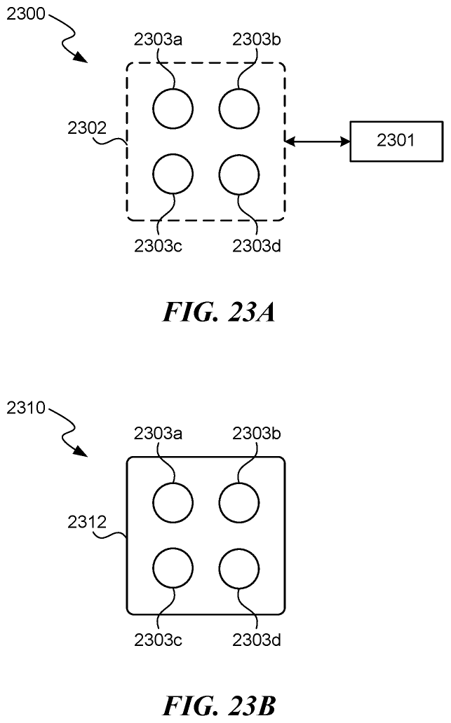

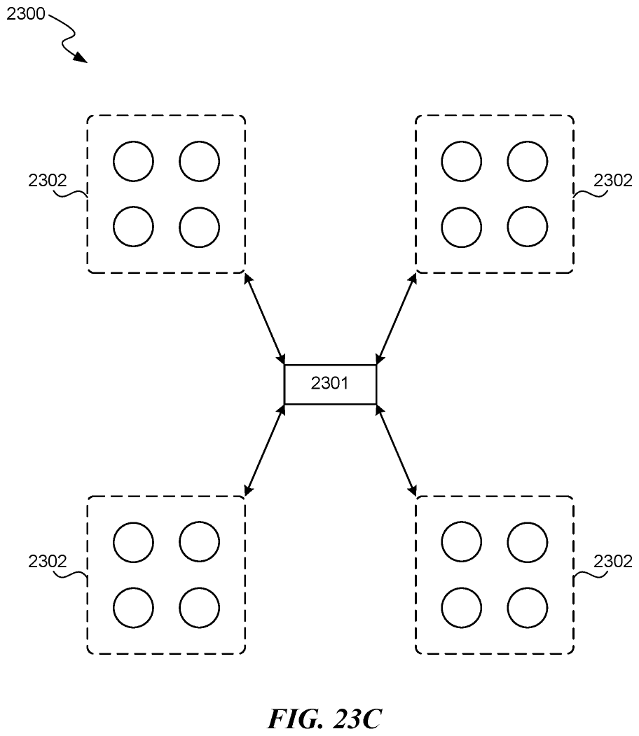

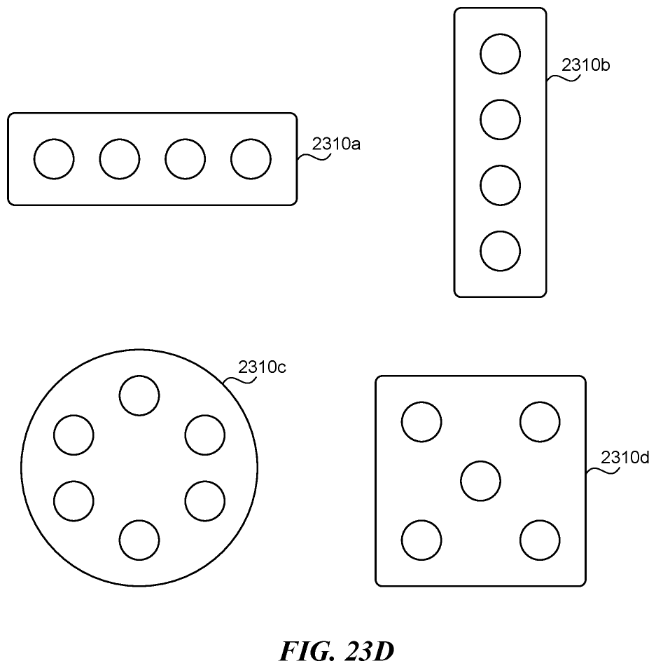

FIG. 23A is a partially schematic view of a rotating stimulus delivery system 2300 configured in accordance with embodiments of the present technology. In the illustrated embodiment, the rotating stimulus delivery system 2300 includes a stimulus group 2302 communicatively coupled to a rotating control station 2301. The stimulus group 2302 includes stimulus elements 2303a-d. The stimulus group 2302 and stimulus elements 2303a-d can communicate with the rotating control station 2301 through any accepted wireless or wired protocol, including radio frequency (RF), infrared light, laser light, visible light, acoustic energy, BLUETOOTH, WIFI, or other communication systems. The rotating control station 2301 can be a desktop or laptop computer, a smartphone, a tablet, or other device. In some embodiments, the control station 2301 can be included with or integrated into a charging station, and/or can share components such as a power source, circuitry, etc., with a charging station. The rotating control station 2301 can instruct the stimulus elements 2303a-d in the stimulus group 2302 to apply heat, electric stimuli, vibration, or other stimulus or combination of stimuli in various patterns to the subject's body.

FIG. 23B illustrates a rotating stimulus device 2310 configured in accordance with embodiments of the present technology. In the illustrated embodiment, the rotation stimulus device 2310 includes the stimulus group 2302 having four stimulus elements 2303a-d arranged spatially proximate to each other. For example, proximate can include distances between each stimulus elements 2303a-d of about 5 inches (12.7 cm), about 3 inches (7.62 cm), about 2 inches (5.08 cm), about 1 inch (2.54 cm), about 0.5 inches (1.27 cm), about 0.25 inches (0.635 cm), or any suitable distance less than 5 inches (12.7 cm). The stimulus elements 2303a-d can be the stimulus pod 110, device 1500, and/or device 1701. The stimulus elements 2303a-d can be attached to a common adhering structure 2312. The adhering structure 2312 can include materials that hold the stimulus elements 2303a-d in place and/or adheres the stimulus elements 2303a-d to a user. In other embodiments, the stimulus elements 2303a-d can be attached to a user without a common physical structure binding them together.

While FIGS. 23A and 23B illustrate the rotating stimulus delivery system 2300 rotating stimulus device 2310 to include one stimulus group 2302 and the stimulus group 2302 includes four stimulus elements 2303, the rotating stimulus delivery system 2300 can include any suitable number of stimulus groups 2302 and each stimulus group 2302 can include any suitable number of stimulus elements 2303. For example, the rotating stimulus delivery system 2300 and rotating stimulus device 2310 can include one, two, three, four, five, ten, or twenty stimulus groups 2302. In a further example, the stimulus group 2302 can include one, two, three, four, five, ten, or twenty stimulus elements 2303. As illustrated in FIG. 23C, rotating stimulus delivery system 2300 can include four stimulus groups 2302. The stimulus elements 2303 can be arranged spatially within the stimulus group 2302 in any suitable fashion. FIG. 23D illustrates several example patterns for the arrangement of the stimulus elements, such as horizontal 2310a, vertical 2310b, circular 2310c, and irregular 2310d, for a rotating stimulus device.

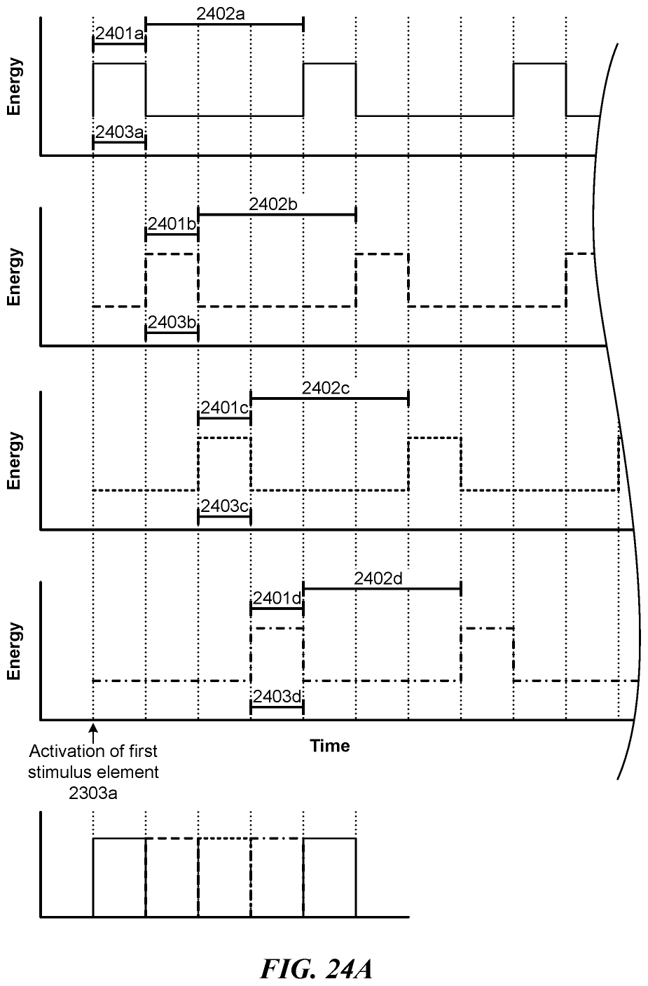

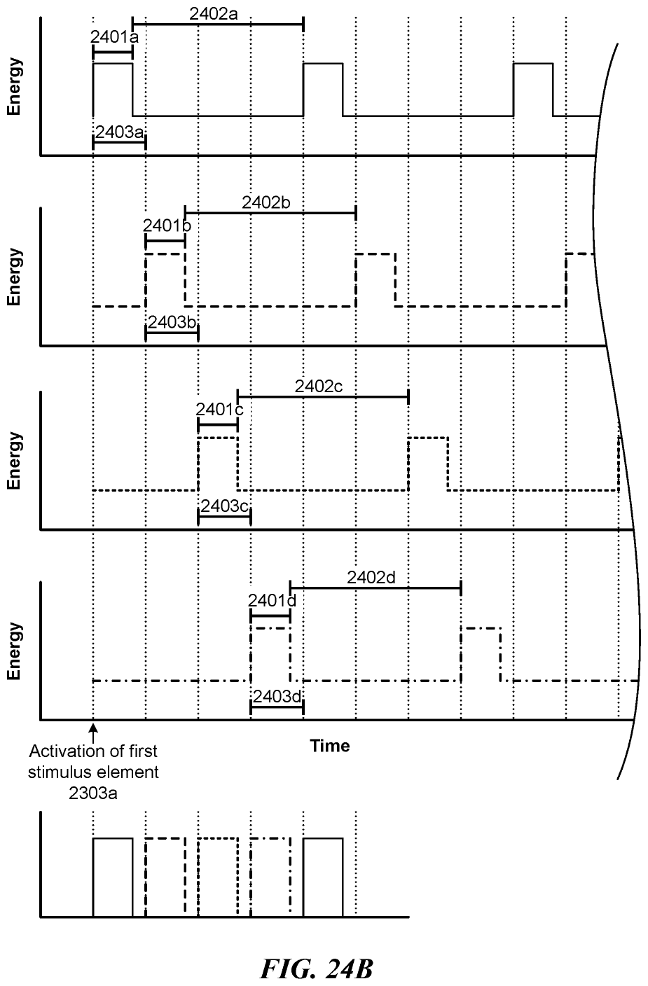

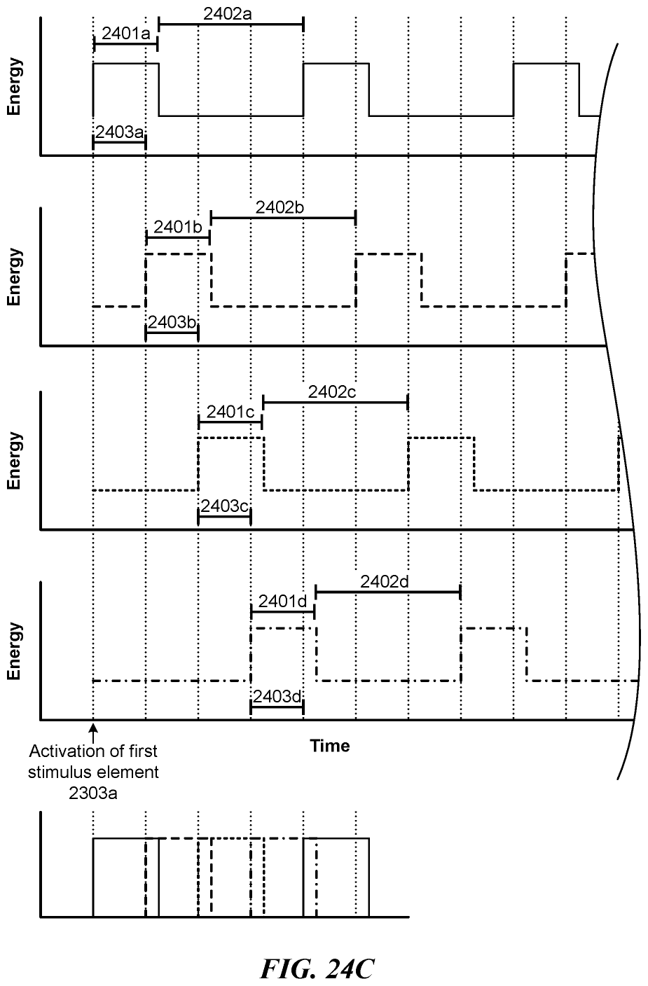

FIGS. 24A-C are graphs illustrating energy application protocols for multiple stimulus elements 2303 in a rotating stimulus delivery system 2300 in accordance with embodiments of the present technology. In the illustrated embodiment, stimulus elements 2302 include four stimulus elements 2303a-d. Each stimulus element 2303a-d is activated, applies a stimulus with an energy level (e.g., temperature) for a hold time 2401a-d, turns off for an off time 2402a-d, and waits a lag time 2403a-d prior to being activated again, respectively. The lag time is the time between activating a subsequent stimulus element in relation to activating a previous stimulus element. For example, upon activation of a first stimulus element 2303a, a second stimulus element 2303b waits a lag time and then is activated. The hold times 2401a-d may be about 4 seconds, about 3 seconds, about 2 seconds, about 1 second, or less than 1 second. The off times 2402a-d may be about 4 seconds, about 3 seconds, about 2 seconds, about 1 second, or less than 1 second. The lag times 2403a-d may be about 4 seconds, about 3 seconds, about 2 seconds, about 1 second, or less than 1 second. Each of the hold times 2401a-d, off times 2402a-d, and lag times 2403a-d can be the same or different. For example, the hold time 2401a can be 1 second, the hold time 2401a can be 2 seconds, the off time 2402a can be 1 second, the off time 2402b can be 3 seconds, the lag time 2403a can be 1 second, and the lag time 2403b can be 2 seconds. In another example, each of the hold times 2401a-d, off times 2402a-d, and lag times 2403a-d can be 1 second.

As illustrated in FIG. 24A, each of the stimulus elements 2303b-d is activated at the beginning of the off time of the previous stimulus element (i.e., the lag time for the activation of each subsequent stimulus element is approximately equal to the hold time of the previous stimulus element). For example, the first stimulus element 2303a is activated, the second stimulus element 2303b is activated when the first stimulus element 2303a is turned off, the third stimulus element 2303c is activated when the second stimulus element 2303b is turned off, and the fourth stimulus element 2303d is activated when the third stimulus element 2303c is turned off.

FIG. 24B illustrates another embodiment of the activation of stimulus elements 2303a-d. Each of the stimulus elements 2303a-d is activated after the end of the hold time of the previous stimulus element (i.e., the lag time for the activation of each subsequent stimulus element is greater than the hold time of the previous stimulus element). For example, the first stimulus element 2303a is activated, the second stimulus element 2303b is activated after the first stimulus element 2303a is turned off, the third stimulus element 2303c is activated after the second stimulus element 2303b is turned off, and the fourth stimulus element 2303d is activated after the third stimulus element 2303c is turned off.

FIG. 24C illustrates another embodiment of the activation of stimulus elements 2303a-d. Each stimulus elements 2303a-d is activated after the activation of the previous stimulus element but before the end of the hold time of the previous stimulus element (i.e., the lag time for the activation of each subsequent stimulus element is less than the hold time of the previous stimulus element). For example, the first stimulus element 2303a is activated, the second stimulus element 2303b is activated after first stimulus element 2303a is activated but before the first stimulus element 2303a is turned off, the third stimulus element 2303c is activated after second stimulus element 2303b is activated but before the second stimulus element 2303b is turned off, and the fourth stimulus element 2303d is activated after third stimulus element 2303c is activated but before the third stimulus element 2303c is turned off.

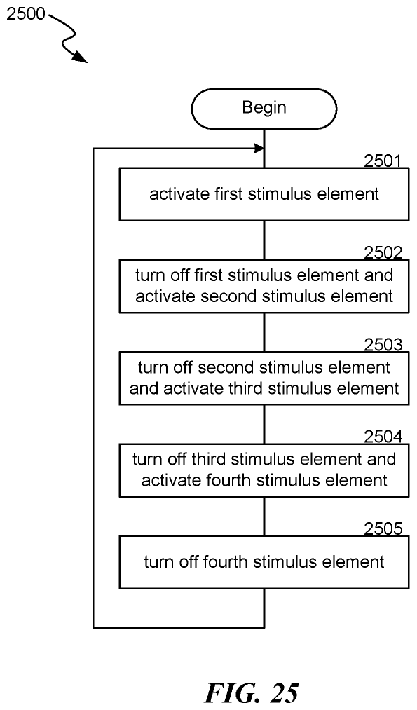

FIG. 25 is a flow diagram of a method 2500 of activating the stimulus delivery system 2300 in accordance to an embodiment of the present technology. In method 2500, the lag times 2403a-d is approximately equal to the hold times 2401a-d, respectively, as described in FIG. 24A. At block 2501, the method 2500 activates the first stimulus element 2303a to apply a stimulus for a first hold time 2401a. At block 2502, the method 2500 turns off the first stimulus element 2303a (i.e., no stimulus is applied) for a first off time 2402a and simultaneously activates the second stimulus element 2303b to apply a stimulus for a second hold time 2401b. At block 2503, the method 2500 turns off the second stimulus element 2303b for a second off time 2402b and simultaneously activates the third stimulus element 2303c to apply a stimulus for a third hold time 2401c. At block 2504, method 2500 turns off the third stimulus element 2303c for a third off time 2402c and simultaneously activates the fourth stimulus element 2303d to apply a stimulus for a fourth hold time 2401d. At block 2505, the method 2500 turns off the fourth stimulus element 2303d for a fourth off time 2402d and returns to block 2501 to activate the first stimulus element 2303a. The method 2500 can cycle through blocks 2501 to 2505 through a number of cycles. For example, the number of cycles can be one, two, three, four, five, ten, twenty, hundreds, thousands, or more times.

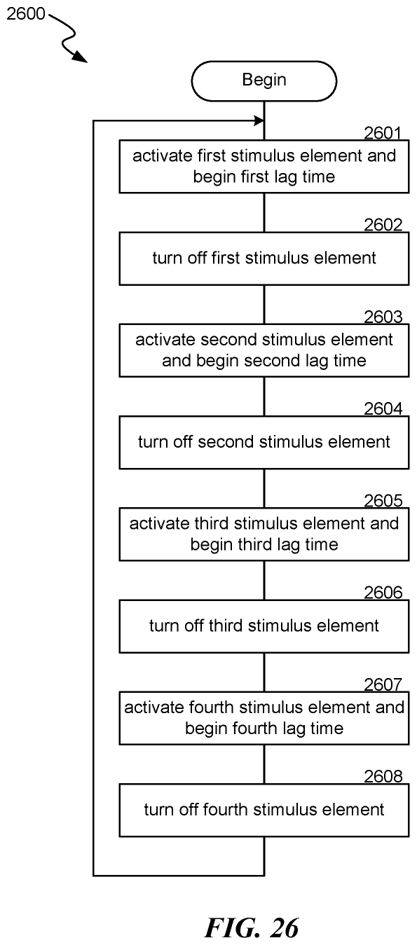

FIG. 26 is a flow diagram of a method 2600 of activating the stimulus delivery system 2300 in accordance to an embodiment of the present technology. In method 2600, the lag times 2403a-d is greater than the hold times 2401a-d, respectively, as described in FIG. 24B. At block 2601, the method 2600 activates the first stimulus element 2303a to apply a stimulus for the first hold time 2401a and begins the first lag time 2403a. At block 2602, the method 2500 turns off the first stimulus element 2303a (i.e., no stimulus is applied) for a first off time 2402a. The end of the first lag time 2403a activates block 2603. At block 2603, the method 2600 activates the second stimulus element 2303b to apply a stimulus for the second hold time 2401b and begins the second lag time 2403b. At block 2604, the method 2600 turns off the second stimulus element 2303b for the second off time 2402b. The end of the first lag time 2403b activates block 2605. At block 2605, the method 2600 activates the third stimulus element 2303c to apply a stimulus for the third hold time 2401c and waits for the lag time 2403c. At block 2606, method 2600 turns off the third stimulus element 2303c for the third off time 2402c. The end of the lag time 2403c activates block 2607. At block 2607, the method 2600 activates the fourth stimulus element 2303d to apply a stimulus for the fourth hold time 2401d and begins the fourth lag time 2403d. At block 2608, the method 2600 turns off the fourth stimulus element 2303d for the fourth off time 2402d. At the end of the fourth lag time 2303d, the method 2600 returns to block 2601 to activate the first stimulus element 2303a. The method 2600 can cycle through blocks 2601 to 2608 through a number of cycles. For example, the number of cycles can be one, two, three, four, five, ten, twenty, hundreds, thousands, or more times.

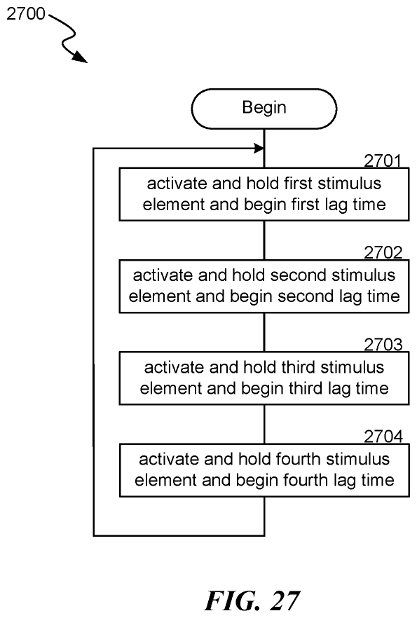

FIG. 27 is a flow diagram of a method 2700 of activating the stimulus delivery system 2300 in accordance to an embodiment of the present technology. In method 2600, the lag times 2403a-d is greater than the hold times 2401a-d, respectively, as described in FIG. 24C. At block 2701, the method 2700 activates the first stimulus element 2303a to apply a stimulus for the first hold time 2401a and begins the first lag time 2403a that is less than the first hold time 2401a. At block 2702, the method 2700 activates the second stimulus element 2303b to apply a stimulus for the second hold time 2401b and begins the second lag time 2403b that is less than the second hold time 2401b. At block 2703, the method 2700 activates the third stimulus element 2303c to apply a stimulus for the third hold time 2401c and begins the third lag time 2403c that is less than the third hold time 2401c. At block 2704, the method 2700 activates the fourth stimulus element 2303d to apply a stimulus for the fourth hold time 2401d and begins the fourth lag time 2403d that is less than the fourth hold time 2401d. At the end of the fourth lag time 2303d, the method 2700 returns to block 2701 to activate the first stimulus element 2303a. The method 2700 turns off the stimulus elements 2303a-d at the end of the hold times 2401-d, respectively. The method 2700 can cycle through blocks 2601 to 2604 through a number of cycles. For example, the number of cycles can be one, two, three, four, five, ten, twenty, hundreds, thousands, or more times.

### XII. SELECTED EMBODIMENTS OF IMPLANTABLE STIMULATION DEVICES

Humans have long used heat to provide pain relief and comfort with many noting that a flare up of back or muscle pain is significantly improved by a hot shower or soak. For many, a hot shower after a workout is a comforting indulgence. Many remain in the shower well past the time needed to wash off the sweat of a workout. The water cleans but the heat reduces pain, spasms, and stress providing a level of comfort and well-being, an oasis in everyday life. In an effort to better understand the mechanism of action (MOA) of thermally induced pain relief, the company, Soovu Labs Inc., has extensively studied this MOA. The company has recently published two successful clinical studies and has a technical paper in submission. (Chabal C, Dunbar P J, Painter I, Young D, and Chabal D C. Properties of Thermal Analgesia in a Human Chronic Low Back Pain Model. Journal of Pain Research 2020a: 13 2083-2092; Chabal C, Dunbar P, Painter I. Is Thermal Analgesia, Exploring the Boundary Between Pain Relief and Nociception Using A Novel Pulsed Heating Device. Anesth Pain Res. 2020b; 4(1): 1-7.) The studies are discussed in greater detail in subsequent sections but reinforce the profound effect that heat has on TRPV1 channels in terms of pain relief. TRPV 1 channels or receptors are widely distributed through the body. The areas of particular interest are TRPV channels located on peripheral nerves, proximal mixed types of nerves, the dorsal root ganglia, and areas of the spinal cord and brain. TRPV1 receptors can be stimulated on the peripheral nerves and tissues, or on central TRPV channels located on larger peripheral nerves, proximal mixed nerves, the dorsal root ganglia, and areas of the spinal cord and brain.

1. Selected Embodiments of Transient Receptor Potential Channels, Vanilloid Subtype (TRPV) as Sensory Mediators

The transient receptor potential cation channels (TRPV) were first identified in Drosophila in 1969. (Cosens D J, Manning A (October 1969). “Abnormal electroretinogram from a Drosophila mutant”. Nature. 224 (5216): 285-7. doi:10.1038/224285a0.) There are six broad classifications of TRPV channels ranging from TRPV1 through TRPV6. TRPV are ion channels and are found in animals and humans. In humans they are widely distributed throughout the body in cells and tissues like the brain, lungs heart, spleen, kidney, placenta and peripheral nervous system. The channel most relevant to pain relief in humans is the TRPV1 channel. TRPV1 are located on afferent sensory fibers, C and Aδ, the dorsal root ganglia as well as multiple other locations in the body. With depolarization, there is an influx Na+ and Ca+ across the cell membrane leading to cellular depolarization and the initiation of an action potential. This action potential can initiate the nociceptive process and drive painful sensations. TRPV1 channels are involved in multiple pain conditions like visceral inflammatory, neuropathic, migraine, and some forms of cancer related pain. (Mickle, A. D., Shepherd, A. J., and Mohapatra, D. P. (2016). Nociceptive TRP channels: sensory detectors and transducers in multiple pain pathologies. Pharmaceuticals 9:E72. doi: 10.3390/ph9040072.) There has been an extensive search for TRPV1 agonists, but success has been limited as TRPV1 channels are also involved with temperature homeostasis which could cause dangerous side effects.

Currently the most commonly used TRPV1 activator is topical capsaicin. While initially the mechanism of action was thought to be depletion of substance P, more recent research indicates that capsaicin works by desensitization of nociceptive fibers in a process known as “defunctionalization.” (Anand P., Bley K. Topical capsaicin for pain management: therapeutic potential and mechanisms of action of the new high-concentration capsaicin 8% patch BJA: British Journal of Anaesthesia, Volume 107, Issue 4, October 2011, Pages 490-502, https://doi.org/10.1093/bja/aer260). This defunctionalization of the TRPV1 channel leads to prolonged block of action potentials and may actually reduce the longer term transport of neurotrophic substances. While initially approved for post herpetic pain, capsaicin is now approved for diabetic neuropathy. TRPV1 channels may be involved with the transmission and maintenance of other chronic pain states. In summary, activation of TRPV1 channels lead to an action potential that can initiate pain impulses. Once activated or depolarized, the channel may become defunctionalized thereby blocking or reducing ongoing depolarization and generation of new action potentials thereby reducing pain sensations.

2. Selected Embodiments of Heating a Selective TRPV1 Activator and Defunctionalization

As set forth above, clinical studies demonstrate that heat is an effective treatment for both acute and subacute pain. In addition, heat increases muscle blood flow and reduces muscle and tendon spasm. Recently, a pair of studies by the Applicant demonstrate that heat is an effective treatment in subjects with longstanding chronic low back pain. In these studies, heat that pulsed to 45° C. at the rate of 2 pulses per minute, produced a rapid onset of pain relief, within 5 minutes. In addition, 30 minutes of treatment reduced pain for over 120 minutes post treatment, compared to subjects who received steady state heat at 37° C. In addition, a subset of subjects who received twice as much thermal energy (four pulses per minute 45° C. versus 2 pulses per minute) showed an increase in pain relief as well as improved pain relief out to 180 minutes after the cessation of treatment. The results demonstrated that high levels of pulsed heat provided the onset of thermally induced pain relief within 5 minutes: further, there was a dose response relationship between the amount of thermal energy delivered and effectiveness including the duration of pain relief from a single 30-minute treatment session.

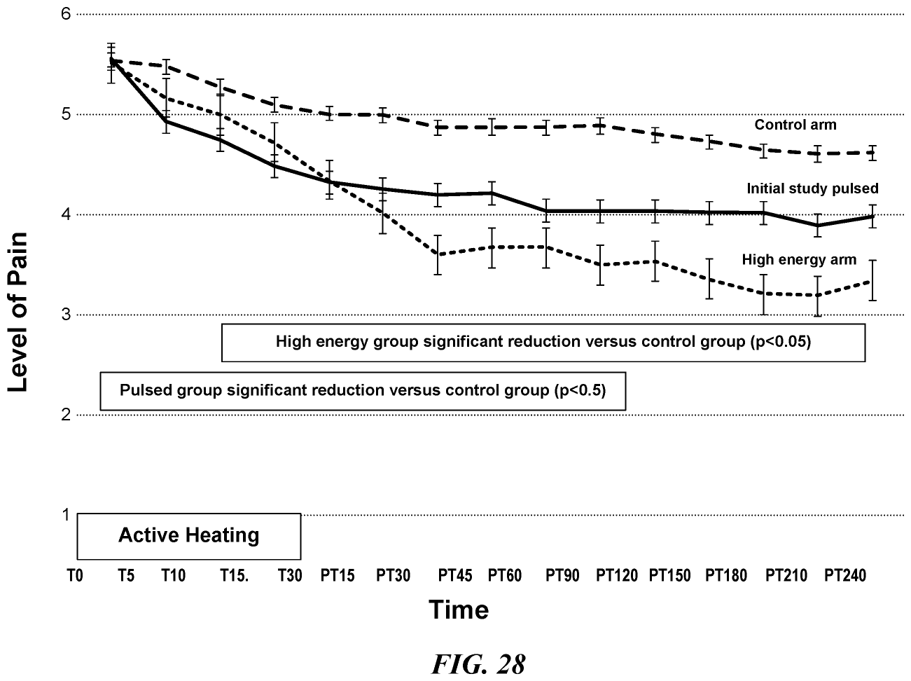

The results of these studies are summarized in FIG. 28. FIG. 28 illustrates a reduction in pain from baseline for control arm (37° C. in yellow), pulsed heat arm (labeled as “initial study pulsed”, 45° C. at 2 pulses/minute in red), and high energy arm (45° C. at 4 pulses/minute in blue) from time T=0 to time T=0 through time T=30 minutes. Shown on the y-axis is a level of pain. As shown, both the pulsed heat and high energy arm rapidly reduced pain and were significantly improved compared to the control arm. The high energy arm sustained a greater reduction of pain compared to the pulsed heat arm. The pulsed heat arm reduced pain for 150 minutes and the high energy arm for 210 minutes.

3. Selected Embodiments of Pulsed High-Level Heat

The rapid onset and long duration of pain relief to applied heat offers some insight into potential mechanisms of action. The range of thermal activation of TRPV1 channels lies in a range from 37° C. to approximately 48° C. Some of this variation is likely due to the method by which TRPV1 channel activation was measured. For example, a recent study, using a cell patch technique demonstrated that activation of TRPV1 channels were temperature dependent. (Sánchez-Moreno A, Guevara-Hernández E, Contreras-Cervera R, et al. Irreversible temperature gating in trpv1 sheds light on channel activation. Elife. 2018; 7:e36372. Published 2018 Jun. 5. doi:10.7554/eLife.36372.) At about 40° C. there was increased sensitivity and a rapid depolarization of these cells. It is expected that in vivo skin temperatures in the range of 43° C. will stimulate TRPV1 channels. This may explain why the heat pulses at 45° C. in the cited articles by Soovu Labs caused such a rapid and profound analgesic response. (Chabal et al. 2020a, b.) In the second study, that delivered twice the amount of thermal energy, the degree of analgesia delivered, and the duration of the response was greater than that delivered with the less frequently pulsed heat arm. If one visualizes intact human skin and underlying tissue, one can think of a thermal pulse radiating out in a three-dimensional cone to penetrate to wider deeper layers of the skin with activation of more TRPV1 channels. A higher energy pulse at four pulses per minute in contrast to two pulses per minute delivers twice the amount of thermal energy and the volume of tissue stimulated would be significantly greater in the higher energy group thereby recruiting more TRPV1 channels. This is a possible explanation why the high energy group had significantly greater analgesic results than the lower energy group. This may also help explain why low-level steady heat from chemical hot packs at 40° C. require up to three hours to provide analgesia. In addition, it is not known if such low-level heat provides lasting pain relief as demonstrated in the high-level pulsed heat experiment.

The high-level pulsed heat results suggest that there is the possibility of even better analgesia than seen in the previous studies. (Chabal et al. 2020a, b.) The upper range or ceiling of thermally induced analgesia where increasing levels of thermal stimulation provide increasing and longer durations of pain relief is limited by an energy point where the heat causes nociception or tissue damage. This can be termed the analgesic nociceptive boundary (ANB). In the pulsed heat studies, the increased energy produced better and longer lasting analgesia without any suggestion of discomfort or tissue damage indicating that the analgesic nociceptive boundary was not reached and providing the opportunity to enhance this analgesia response in the future. Additional pulses of energy during the extended period of pain relief could further prolong the duration of relief.

The use of pulsed high-level heat can be chosen based on a number of factors. It is generally accepted that the selective agonist of TRPV1, capsaicin, binds to the TRPV1 channel and can cause prolonged deactivation. In the case of high concentration of capsaicin that deactivation combined with the loss of some terminal branches of C fibers can produce analgesia lasting up to three months. Heat may also be thought of a selective TRPV1 agonist with some studies showing that a brief dose of high-level heat can produce prolonged pain relief that greatly exceeds the actual duration of heat. This concept is reinforced by the recent study by Sánchez-Moreno et al 2018. In this study the effect of heat on TRPV1 channels was observed using a cell patch technique. Relatively short durations of heat in the mid 40° C. range produced irreversible depolarization of the TRPV1 receptor. The clinical studies using high level pulsed heat are likely related to the prolonged deactivation of the TRPV1 channel thereby producing a period of prolonged analgesia.

In some embodiments, a series of high-level heat pulses can produce sensitization of the TRPV channel whereas subsequent thermal pulses can produce greater depolarization of the channel. As noted, a series of thermal pulses sensitize the channel to the point that a thermal pulse at the same temperature produces a significantly greater action potential. Finally, pulsed heat stimulation can provide significant thermal stimulation yet minimized the amount of energy transferred to the skin offering increased safety.

The use of high level pulsed heat in the clinical studies described by Chabal et al, 2020 a, b, were based on laboratory data showing that a series of pulses at constant temperature sensitize the TRPV1 channel so that subsequent thermal pulses produce a significantly enhanced response and that this response maybe prolonged or even irreversible as demonstrated by Sánchez-Moreno et al 2018. Based on these data it was hypothesized that the application of thermal energy approaching the analgesic nociceptive boundary would heat a greater volume of subcutaneous tissue resulting in the stimulation greater numbers of TRPV1 channels therefore producing greater and longer lasting analgesia. The clinical results support this hypothesis.

4. Summary and Implications for Improved Clinical Care

Applied localized heat provides significant pain relief in both acute and chronic pain conditions. This is based on a number of well-designed clinical trials. The advantage of heat is that it offers a rapid onset, clinically effective, and drug free alternative. In addition, unlike many other therapies, heat provides a sense of comfort and relief often termed the hedonic response.

It is hypothesized that high level heat (>40° C.) activates TRPV1 receptors producing a very rapid and prolonged analgesic response. Basic science and laboratory data provide a foundation for thermal analgesia based on the prolonged deactivation of TRPV1 channels.

Translational clinical research builds on these laboratory concepts and applies them to human physiology. The behavior of TRPV1 channels to high level pulsed heat provides a well-defined target for thermal activation. The pulsing of high-level heat sensitizes TRPV1 channels producing greater activation while reducing the amount of thermal energy required to produce analgesia, thereby increasing patient safety.

The recent studies are one of the first to examine the effect of thermal energy on human chronic pain and offers the concept of the analgesic nociceptive boundary (ANB). The ANB is defined as the amount of thermal energy needed to cause maximal pain relief while not causing nociception or tissue damage. This boundary provides a framework for future clinical development. Exploration and a better understanding of the thermal analgesic boundary offers a model that may result in significant clinical improvement and understanding of thermal analgesia.

Stimulation of C-fibers are associated with changes in the limbic system of the amygdala are under appreciated. Reinforcing stress management techniques with the physical activation of C-fiber afferents offers a potentially powerful synergy by coupling activation of neurological pathways with psychological exercises to reduce pain or anxiety.

5. Selected Embodiments of Pain Relief Devices

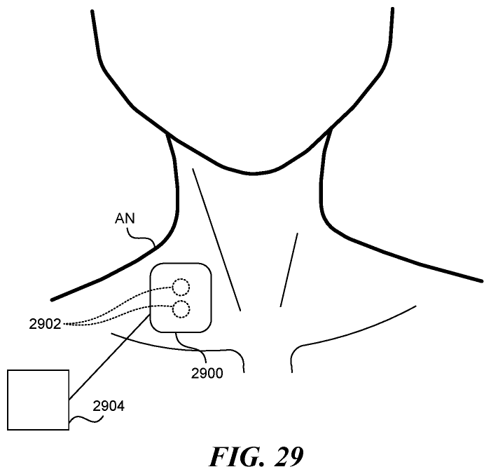

FIG. 29 is a front view of a pain relief device 2900 attached to a human subject and configured to project heat to underlying nerves in accordance with embodiments of the present technology. The pain relief device 2900 can include a patch with an adhesive side and/or other attachment mechanism that adheres or otherwise affixes the patch to a subject's skin. The pain relief device 2900 can include one or more electrodes 2902 that can be activated to provide heat to underlying nerves of the human subject. The one or more electrodes 2902 can be operably coupled to a power source 2904 (e.g., a battery) that powers the one or more electrodes 2902. For example, the power source 2904 may be a separate power device (not shown) operably coupled to the one or more electrodes 2902 via a wired or wireless connection, and/or the energy source may be positioned within the patch of the pain relief device 2900. In the illustrated embodiment, the pain relief device 2900 is positioned near or above the collar bone, close to or along the human subject's neck, and therefore the heat generated by the pain relief device 2900 can target the brachial nerves and plexus in the anterior neck AN. In some embodiments, the pain relief device 2900 can be positioned elsewhere on the human subject to target different nerves.

In some embodiments, the power source 2904 may communicate and power an implanted thermal stimulator having features generally similar to the pain relief device 2900, but instead of external attachment, the implanted thermal stimulator is implanted under the skin to produce heat affecting the nerves near the implanted thermal stimulator. In some embodiments, the pain relief device 2900 can include features generally similar to or the same as the stimulus pod 110 and/or hedonic device 1500 described above with reference to FIGS. 1A-15B.

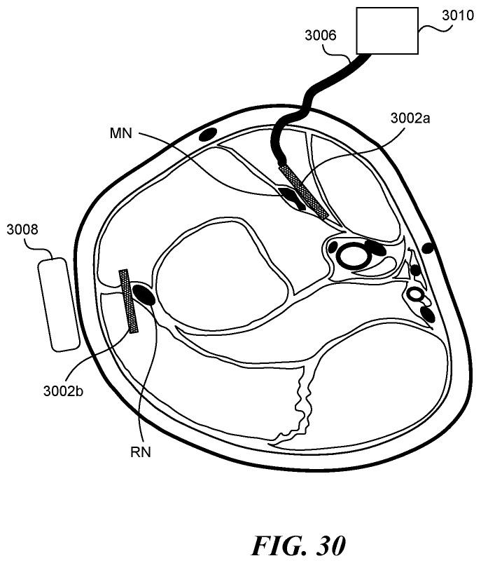

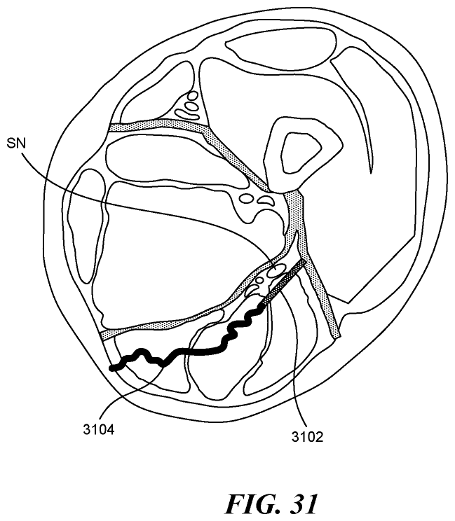

FIG. 30 is a transverse view of electrodes (identified individually as a first electrode 3002a and a second electrode 3002b; referred to collectively as “the electrodes 3002”) implanted in an arm of a human subject in accordance with embodiments of the present technology. The electrodes 3002 provide heat to one or more nerves in the arm of the human subject. The electrodes 3002 can deliver heat in the form of pulsed heat, continuous heat, and/or in varying different patterns. As shown in FIG. 30, the first electrode 3002a can be placed over the musculocutaneous nerve MN, and the second electrode 3002b can be placed over the radial nerve RN such that the heat generated by the electrodes 3002 can affect the musculocutaneous nerve MN and the radial nerve RN. In these and other embodiments, one or more electrodes 3002 can be implanted in the arm of the human subject and placed other suitable nerves in the arm of the human subject.

The electrodes 3002 can be operably coupled to a power source via a wired or wireless connection. For example, as shown in FIG. 30, the first electrode 3002a is connected to a wired power source 3010 via a tunneled wire 3006. The wired power source 3010 can be positioned elsewhere in the body (e.g., in a subcutaneous pocket) or external to the skin. As further shown in FIG. 30, the second implanted electrode 3002b can be connected to a wireless power source 3008 to provide wireless power transfer known to persons skilled in the art, including capacitive coupling, inductive coupling, magnetic resonance coupling, radiative coupling, acoustic coupling, and/or optical coupling. The power source 3008 may be implanted subcutaneously or externally placed over or near the electrode 3002b (e.g., as shown in FIG. 30).

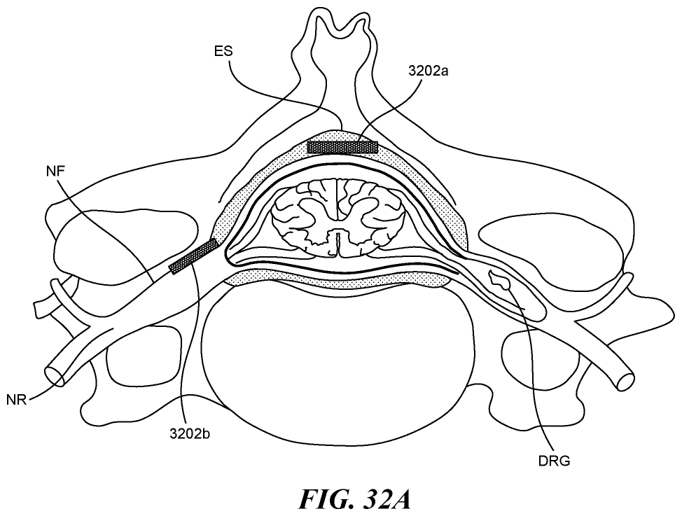

FIG. 31 is a transverse view of an implantable electrode 3102 positioned in an upper leg of a human subject in accordance with embodiments of the present technology. The implantable electrode 3102 provides heat to one or more nerves in the upper leg of the human subject, such as the sciatic nerve SN. The implantable electrode 3102 can deliver heat in the form of pulsed heat, continuous heat, or in different patterns. In the illustrated embodiments, the implantable electrode 3102 can be connected to a power source and/or a controller (not illustrated) via a subcutaneous wire 3104. In other embodiments, the implantable electrode 3102 can be connected to the power source and/or controller via wireless means as described above with respect to FIG. 30.

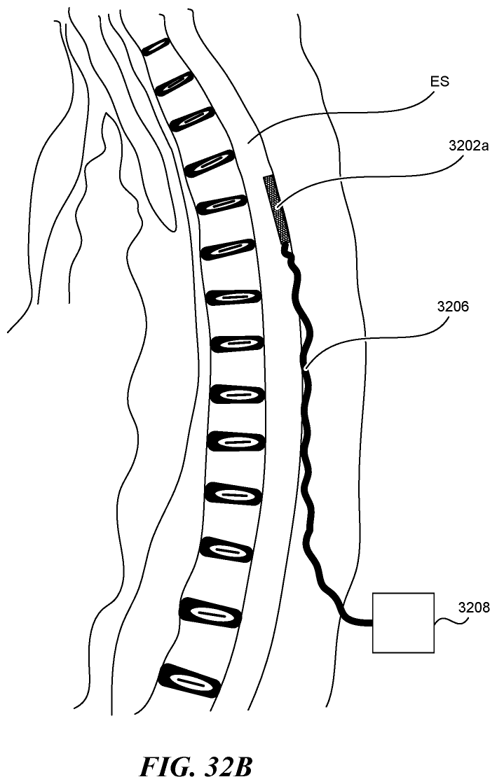

FIG. 32A is a transverse view of a stimulus system including multiple electrodes (identified individually as a first electrode 3202a and a second electrode 3202b; referred to collectively as “the implanted electrodes 3202”) implanted along a spinal cord of a human subject in accordance with embodiments of the present technology, and FIG. 32B is a longitudinal view of the first electrode 3202a implanted along the spinal cord of the human subject. The implanted electrodes 3202 provide heat to the epidural space ES and/or to one or more nerves in the spinal cord of the human subject. The implanted electrodes 3202 can deliver heat in the form of pulsed heat, continuous heat, or in different patterns. As shown in the illustrated embodiment, the first electrode 3202a can be implanted in the epidural space ES, while the second electrode 3202b can be placed in or proximate to the neural foramen NF to provide heat to the nerve root NR and dorsal root ganglion DRG. In some embodiments, one or more of the implanted electrodes 3202 can be positioned at additional locations in the epidural space ES or the neural foramen NF, and/or one or more of the electrodes 3202 can be positioned elsewhere proximate to the spinal column.

Referring to FIG. 32B, one or more of the implanted electrodes 3202 can be connected to a power source and/or controller 3208 via wired and/or wireless means as described above with respect to FIG. 30. In the embodiment illustrated in FIG. 32B, for example, the first electrode 3202a has a connecting wire 3206 that couples to an externally positioned power source and/or controller 3208.

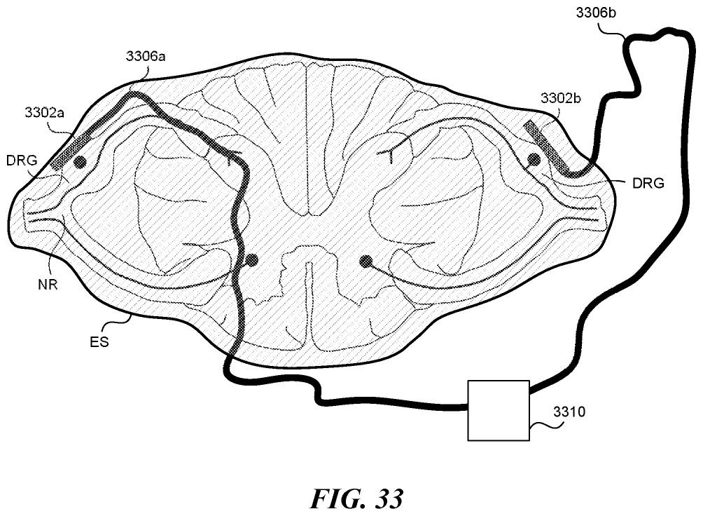

FIG. 33 is a transverse view of a system including multiple electrodes (identified individually as a first electrode 3302a and a second electrode 3302b; referred to collectively as “the implanted electrodes 3302”) implanted along a spinal cord of a human subject in accordance with embodiments of the present technology. Similar to the implanted electrodes 3202 of FIGS. 32A and 32B, the implanted electrodes 3302 are positioned near nerves at or proximate to the spinal column to provide heat to one or more nerves in the spinal cord of the human subject. In the illustrated embodiment, the implanted electrodes 3302 are placed over the dorsal root ganglion DRG and connected to a power source and/or controller 3310 via connecting wires (identified individually as a first connecting wire 3306a and a second connecting wire 3306b; referred to collectively as “the connecting wires 3306”). As shown in FIG. 33, the first connecting wire 3306a traverses the epidural space ES, and the second connecting wire 3306b is positioned external to the epidural space ES. In various embodiments, the connecting wires 3306 of multiple electrodes 3302 can extend through the epidural space ES, the connecting wires 3306 of multiple electrodes 3302 can be positioned exclusively external to the epidural space ES, and/or one or more of the implanted electrodes 3202 can be connected to the power source and/or controller 3310 via wireless means. Although FIG. 33 illustrates two implanted electrodes 3302 over the dorsal root ganglion DRG, one or more than two implanted electrodes 3302 can be placed over the dorsal root ganglion and/or other suitable nerves along the spinal cord.

In some embodiments, systems disclosed herein can include multiple implantable electrodes (e.g., two or more of the electrodes 2902, 3002, 3102, 3202, 3302) positioned beneath the skin along one or more locations within a human subjects, arm, leg, torso, and/or other target sites to provide heating to proximal neural fibers for pain relief and/or other indications. In various embodiments, systems disclosed herein can include one or more implantable electrodes and one or more externally positioned stimulus devices (e.g., the stimulus pods 110 of FIGS. 1A-4, the hedonic device 1500 of FIGS. 15A and 15B, the stimulus devices 2010 of FIG. 20, the rotating stimulus delivery system 2300 of FIGS. 23A-23D, and the patch-type stimulus device 2900 of FIG. 29), each positioned at a target location within the body or on the skin to provide heating and/or other stimuli to target neural fibers.

### XIII. EXAMPLES

The present technology may be better understood with reference to the following non-limiting examples.

1. A method of activating a hedonic response of a human subject, the method comprising:
applying pulsed energy, via a stimulus device, into a volume of tissue including thermoreceptors;
detecting, via a monitoring device, a physiological parameter of the human subject during application of the pulsed energy;
associating, via a control unit, the detected physiological parameter to a reference parameter indicative the hedonic response; and
synchronizing treatment parameters of the stimulus device with the detected physiological parameter to activate the hedonic response.

2. The method of example 1 wherein applying pulsed energy into the volume of tissue comprises applying pulsed energy at an intensity and duration to activate nerve fibers in the volume of tissue.

3. The method of example 2 wherein the nerve fibers include A-delta fibers and/or C-fibers.

4. The method of example 1 wherein applying pulsed energy comprises pulsing heat.

5. The method of example 1 wherein applying pulsed energy comprises pulsing radiant heat.

6. The method of example 1 wherein applying pulsed energy comprises pulsing heat between 40 degrees Celsius and 60.5 degrees Celsius.

7. The method of example 4 wherein pulsing heat into the volume of tissue comprises executing at least one heat cycle including a ramp-up phase, a hold phase, a release phase, and a soak phase.

8. The method of example 7 wherein the hold phase is within a range of 0.5 seconds to 120 minutes.

9. The method of example 1 wherein applying pulsed energy comprises applying heat to a continuous surface of the tissue having an area of at least 1.27 cm².

10. The method of example 1 wherein applying pulsed energy comprises pulsing at least one of thermal energy, vibrational energy, mechanical energy, pressure energy, electrical energy, or ultrasound energy.

11. The method of example 1 wherein detecting the physiological parameter includes measuring brain wave activity, skin resistance, heart rate, breathing, respiratory pattern, respiratory variability, muscle tension, blood flow, body temperature, oxygenation, and/or temperature trigger stimulation of a heat receptor, mechanical receptor, and/or electrical pulses.

12. The method of example 1 wherein detecting the physiological parameter comprises using electromyography (EMG) and/or electroencephalography (EEG).

13. The method of example 1 wherein applying pulsed energy in accordance with the treatment parameters is configured to alter an emotional state of the subject.

14. The method of example 1 wherein applying pulsed energy in accordance with the treatment parameters is configured to trigger a sensation akin to stroking and/or moving touch in the human subject.

15. The method of example 1 wherein applying pulsed energy in accordance with the treatment parameters is configured to modulate breathing, breathing pattern, heart rates, blood pressure, and/or brain wave activity of the human subject.

16. The method of example 1 wherein applying pulsed energy comprises pulsing vibrational energy to modulate breathing, breathing patterns, heart rate, blood pressure, and/or brain wave activity of the human subject.

17. The method of example 1 wherein applying pulsed energy in accordance with the treatment parameters is configured to dilate blood vessels of the human subject and/or enhance an immune system response of the human subject.

18. The method of example 1 wherein applying pulsed energy into the volume of tissue comprises pulsing energy into smooth muscles to treat chronic disorders.

19. The method of example 1 wherein the stimulus device is affixed to skin of the human subject, and wherein applying pulsed energy into the volume of tissue comprises applying pulsed heat, via the stimulus device, to the skin.

20. The method of example 1 wherein the stimulus device is an implantable device, and wherein applying pulsed energy into the volume of tissue comprises applying pulsed heat, via the stimulus device, from a location within the human subject.

21. The method of example 1 wherein applying pulsed heat comprises applying pulsed heat from the stimulus device positioned at a location within an arm of the human subject, within a leg of the human subject, and/or proximate to a spinal cord of the human subject.

22. A method of increasing blood flow of a subject to reduce muscle activity and tension, the method comprising:
applying pulsed heat, via a stimulus device, into a volume of muscle tissue of the human subject;
applying continuous heat, via the stimulus device, into the volume of muscle tissue of a human subject;
measuring, via a monitoring device, a physiological parameter of the human subject during application of the continuous heat and/or the pulsed heat; and
defining treatment parameters of the stimulus device such that the applied continuous heat and the pulsed heat synchronize with the measured physiological parameter to stimulate nerve fibers and thermoreceptors to reduce muscle activity and tension.

23. The method of example 22 wherein:
applying pulsed heat comprises applying the pulsed heat for a first period at a first temperature range, the first temperature range having a maximum temperature; and
applying continuous heat comprises applying continuous heat for a second period at a second temperature, wherein the second period is after the first period, and wherein the second temperature is above 40° C. and less than the maximum temperature.

24. The method of example 22 wherein applying pulsed heat comprises pulsing heat between 40 degrees Celsius and 60.5 degrees Celsius.

25. The method of example 22 wherein applying pulsed heat into the volume of tissue comprises executing a heat cycle including a ramp-up phase, a hold phase, a release phase, and a soak phase.

26. The method of example 25 wherein the hold phase has a duration between 0.5 seconds to 120 minutes.

27. The method of example 22 wherein applying pulsed heat comprises applying heat to a continuous surface of tissue having an area of at least 1.27 cm².

28. The method of example 22 wherein measuring the physiological parameter comprises measuring at least one of blood flow, tissue blood flow, tissue oxygenation, muscle tension, muscle activation, skin temperature, subcutaneous or muscle temperature via direct or inferred measurements, and skin thermal flux.

29. The method of example 22 wherein measuring the physiological parameter is performed while applying pulsed heat.

30. The method of example 22, further comprising receiving, at a control device, user preferences including one or more desired physiological parameters.

31. The method of example 30 wherein defining the treatment parameters comprises defining the treatment parameters based on the user preferences, the treatment parameters comprising temperature and pulse duration, and wherein the method further comprises applying pulsed heat in accordance with the treatment parameters.

32. The method of example 22 wherein the stimulus device is implanted within the human subject, and wherein applying pulsed heat comprises applying pulsed heat from a location within the human subject.

33. A method of affecting a physiological parameter a human subject to reduce pain of the subject, the method comprising:
applying, via a stimulus device, pulsed heat into a volume of tissue of the human subject, wherein the pulsed heat is configured to affect at least one physiological parameter including blood flow, oxygen delivery, muscle tension, or range of motion;
measuring, via a monitoring device, the at least one physiological parameter of the human subject during application of the pulsed heat; and
defining treatment parameters of the stimulus device such that the pulsed heat synchronizes with the measured physiological parameter to reduce pain.

34. The method of example 33 wherein applying pulsed heat comprises pulsing heat between 45 degrees Celsius and 60 degrees Celsius.

35. The method of example 33 wherein applying pulsed heat into the volume of tissue comprises executing a heat cycle including a ramp-up phase, a hold phase, a release phase, and a soak phase.

36. The method of example 35 wherein the hold phase is less than 3 seconds.

37. The method of example 33 wherein applying pulsed heat comprises pulsing heat at regular intervals.

38. The method of example 33 wherein applying pulsed heat comprises pulsing heat at random intervals.

39. The method of example 33 wherein applying pulsed heat comprises pulsing heat having a temperature of at most 45 degrees Celsius at a rate of at least two pulses per minute.

40. The method of example 35 wherein the soak time is within a range of one second to 120 minutes.

41. The method of example 35, further comprising:
receiving, at a control device, user preferences include a number of heat cycles; and
executing the number of heat cycles.

42. The method of example 35, wherein executing the heat cycle comprises applying heat greater than 45 degrees Celsius, the hold phase having a duration of less than 3 seconds.

43. A method of relaxing muscles and ligaments of a human subject, the method comprising:
applying, via a stimulus device, pulsed heat into a volume of tissue proximate to a joint of the human subject;
measuring, via a stimulus device, a physiological parameter of the human subject; and
defining treatment parameters of the stimulus device such that the pulsed heat synchronizes with the measured physiological parameter to increase a range of motion around the joint.

44. The method of example 43 wherein the joint is at least one of: a) a major axial join or minor axial joint including at least one of knees, shoulders, hips, ankle, elbow, wrist, fingers, toes, jaw, or areas of the back; b) a joint contained in neuraxial structures of a first cervical vertebrate through a last lumbar vertebra; or c) a sacral joint.

45. The method of example 43 wherein applying pulsed heat comprises pulsing heat into the volume of tissue prior to a physical activity and/or physical therapy by the human subject.

46. The method of example 43 wherein measuring the physiological parameter comprises measuring the physiological parameter with at least one of an electromyography or a goniometer.

47. The method of example 43 wherein the stimulus device is one of a plurality of stimulus devices, and wherein applying pulsed heat comprises applying pulsed heat via the multiple stimulus devices at multiple target sites on the human subject.

48. A method of suppressing an appetite of a human subject, the method comprising:
applying, via a stimulus device, pulsed energy into a volume of tissue including thermoreceptors associated with appetite;
measuring, via a monitoring device, a physiological parameter of the human subject; and
defining treatment parameters of the stimulus device such that the pulsed energy synchronizes with the measured physiological parameter to suppress appetite.

49. The method of example 48 wherein applying pulsed energy comprises pulsing heat in accordance with the treatment parameters to stimulate and/or induce desensitization of TRPV1 receptors located in skin of the human subject.

50. The method of example 48 wherein applying pulsed energy comprises pulsing at least one of thermal, vibrational, electrical, and mechanical energy.

51. The method of example 48 wherein applying pulsed energy comprises pulsing heat between 40 degrees Celsius and 60.5 degrees Celsius.

52. The method of example 48 wherein applying pulsed energy into the volume of tissue comprises executing a heat cycle including a ramp-up phase, a hold phase, a release phase, and a soak phase.

53. The method of example 48 wherein the hold phase has a duration of 0.5 seconds to 120 minutes.

54. The method of example 48 wherein the stimulus device is an implantable device, and wherein applying pulsed energy into the volume of tissue includes applying energy from a target site within the human subject.

55. A pulsed heating system for treating a human subject, the pulsed heating system comprising:
a group of heating elements configured to pulse energy into a volume of tissue of the human subject, the volume of tissue including thermoreceptors;
a control unit operably coupled to the heating elements, wherein the control unit is configured to activate each heating element asynchronously; and
a power source operably coupled to the group of heating elements and the control unit.

56. The system of example 55 wherein the group of heating elements comprises at least a first heating element and a second heating element, and wherein the first heating element is configured to pulse energy into a first volume of tissue and the second heating element is separate from the first heating element and configured to pulse energy into a second volume of tissue spaced apart from the first volume.

57. The system of example 55, further comprising a substrate carrying the group of heating elements, wherein the substrate is configured to attach to skin of the human subject.

58. The system of example 55 wherein each heating element comprises at least one electrode.

59. The system of example 55, further comprising a substrate carrying at least one of the heating elements, and wherein the substrate comprises an adhesive for adhering to skin of the human subject.

60. A pulsed heating system for treating a human subject, the pulsed heating system comprising:
an implantable heating element sized and shaped to be positioned within a volume of tissue of the human subject, the implantable heating element comprising an electrode configured to apply pulsed heat to thermoreceptors proximate to the volume of tissue;
a control unit operably coupled to the implantable heating element and configured to send instructions to the heating element that causes the heating element to apply heat in accordance with a treatment protocol; and
a power source operably coupled to the heating element.

61. The pulsed heating system of example 60 wherein the implantable heating element is configured to be implanted proximate to at least one of an epidural space, dorsal root ganglion, nerves, and a nerve plexus.

62. The pulsed heating system of example 60 wherein the implantable heating element is configured to stimulate and/or induce desensitization of TRPV receptors located beneath skin of the human subject.

63. The pulsed heating system of example 60 wherein the control unit is configured to be implanted beneath skin of the human patient.

64. The pulsed heating system of example 60 wherein the power source is configured to be implanted beneath skin of the human patient.

65. The pulsed heating system of example 60 wherein the implantable heating element is configured to apply heat to at least one of nerve endings, terminal branches of nerves, and nerves including at least one of:
a) axillary nerves and all branches,
b) brachial nerves and plexus,
c) radial, medial and ulnar nerves,
d) sciatic nerves and all branches,
e) femoral nerves, plexus, and all branches,
f) cranial nerves, or
g) spinal nerves including anterior, posterior divisions and distal nerves including at least one of:
h) C1-C8: Cervical nerves,
i) T1-T12: Thoracic nerves,
j) L1-L5: Lumbar nerves including plexus,
k) S1-S5: Sacral nerves,
l) coccygeal nerves, or
m) sympathetic nerves including a vagus nerve.

66. The pulsed heating system of example 60 wherein the implantable heating element is configured to be implanted proximate to dorsal root ganglia and/or nerve roots proximal to the dorsal root ganglia.

67. The pulsed heating system of example 60 wherein the implantable heating element is configured to be implanted at or proximate to at least one of a cauda equina, a spinal cord, and an epidural space.

68. The pulsed heating system of example 60 wherein the implantable heating element is configured to be implanted at or proximate to a brain of the human subject.

69. The pulsed heating system of example 60 wherein the implantable heating element is configured to apply heat between 37 degrees Celsius and 50 degrees Celsius.

70. The pulsed heating system of example 60 wherein the implantable heating element is one of a plurality of implantable heating elements configured to be positioned at target sites within the human subject.

71. The pulsed heating system of example 60 wherein:
the implantable heating element is one of a plurality of implantable heating elements including at least a first implantable heating element and a second implantable heating element; and
the controller device is configured to activate the first implantable heating element independent of the second implantable heating element.

72. The pulsed heating system of example 60 wherein the implantable heating element is configured to apply heat to stimulate the thermoreceptors associated with pain.

### XIV. CONCLUSION

Systems, methods, and devices as described herein can be used in combination with any of the systems, methods, and devices as described in FIGS. 1-33. For example, the stimulus elements 2303a-d can be stimulus pods 110, and the stimulus group 2302 can include one or more stimulus pods 110. The control station 2301 can apply stimulus in the form of pulsed heat, continuous heat, or in different patterns from the stimulus pods 110. In a further example, monitoring devices 1703 can be combined with the stimulus elements 2303a-d to provide feedback data. The hold time 2401, off time 2402, and lag time 2403 can be determined based on the feedback data.

From the foregoing, it will be appreciated that specific embodiments of the invention have been described herein for purposes of illustration, but that various modifications may be made without deviating from the scope of the invention. Accordingly, the invention is not limited except as by the appended claims.

1. A method of activating a hedonic response of a human subject, the method comprising:
applying pulsed energy, via a stimulus device, into a volume of tissue including thermoreceptors;
detecting, via a monitoring device, a physiological parameter of the human subject during application of the pulsed energy;
associating, via a control unit, the detected physiological parameter to a reference parameter indicative the hedonic response; and
synchronizing treatment parameters of the stimulus device with the detected physiological parameter to activate the hedonic response.

2. The method of claim 1 wherein applying pulsed energy into the volume of tissue comprises applying pulsed energy at an intensity and duration to activate nerve fibers in the volume of tissue.

3. The method of claim 2 wherein the nerve fibers include A-delta fibers and/or C-fibers.

4. The method of claim 1 wherein applying pulsed energy comprises pulsing heat.

5. The method of claim 1 wherein applying pulsed energy comprises pulsing radiant heat.

6. The method of claim 1 wherein applying pulsed energy comprises pulsing heat between 40 degrees Celsius and 60.5 degrees Celsius.

7. The method of claim 4 wherein pulsing heat into the volume of tissue comprises executing at least one heat cycle including a ramp-up phase, a hold phase, a release phase, and a soak phase.

8. The method of claim 7 wherein the hold phase is within a range of 0.5 seconds to 120 minutes.

9. The method of claim 1 wherein applying pulsed energy comprises applying heat to a continuous surface of the tissue having an area of at least 1.27 cm².

10. The method of claim 1 wherein applying pulsed energy comprises pulsing at least one of thermal energy, vibrational energy, mechanical energy, pressure energy, electrical energy, or ultrasound energy.

11. The method of claim 1 wherein detecting the physiological parameter includes measuring brain wave activity, skin resistance, heart rate, breathing, respiratory pattern, respiratory variability, muscle tension, blood flow, body temperature, oxygenation, and/or temperature trigger stimulation of a heat receptor, mechanical receptor, and/or electrical pulses.

12. The method of claim 1 wherein detecting the physiological parameter comprises using electromyography (EMG) and/or electroencephalography (EEG).

13. The method of claim 1 wherein applying pulsed energy in accordance with the treatment parameters is configured to alter an emotional state of the subject.

14. The method of claim 1 wherein applying pulsed energy in accordance with the treatment parameters is configured to trigger a sensation akin to stroking and/or moving touch in the human subject.

15. The method of claim 1 wherein applying pulsed energy in accordance with the treatment parameters is configured to modulate breathing, breathing pattern, heart rates, blood pressure, and/or brain wave activity of the human subject.

16. The method of claim 1 wherein applying pulsed energy comprises pulsing vibrational energy to modulate breathing, breathing patterns, heart rate, blood pressure, and/or brain wave activity of the human subject.

17. The method of claim 1 wherein applying pulsed energy in accordance with the treatment parameters is configured to dilate blood vessels of the human subject and/or enhance an immune system response of the human subject.

18. The method of claim 1 wherein applying pulsed energy into the volume of tissue comprises pulsing energy into smooth muscles to treat chronic disorders.

19. The method of claim 1 wherein the stimulus device is affixed to skin of the human subject, and wherein applying pulsed energy into the volume of tissue comprises applying pulsed heat, via the stimulus device, to the skin.

20. The method of claim 1 wherein the stimulus device is an implantable device, and wherein applying pulsed energy into the volume of tissue comprises applying pulsed heat, via the stimulus device, from a location within the human subject.

21. The method of claim 1 wherein applying pulsed heat comprises applying pulsed heat from the stimulus device positioned at a location within an arm of the human subject, within a leg of the human subject, and/or proximate to a spinal cord of the human subject.

22. A method of increasing blood flow of a subject to reduce muscle activity and tension, the method comprising:
applying pulsed heat, via a stimulus device, into a volume of muscle tissue of the human subject;
applying continuous heat, via the stimulus device, into the volume of muscle tissue of a human subject;
measuring, via a monitoring device, a physiological parameter of the human subject during application of the continuous heat and/or the pulsed heat; and
defining treatment parameters of the stimulus device such that the applied continuous heat and pulsed heat synchronize with the measured physiological parameter to stimulate nerve fibers and thermoreceptors to reduce muscle activity and tension.

23. The method of claim 22 wherein:
applying pulsed heat comprises applying the pulsed heat for a first period at a first temperature range, the first temperature range having a maximum temperature; and
applying continuous heat comprises applying continuous heat for a second period at a second temperature, wherein the second period is after the first period, and wherein the second temperature is above 40° C. and less than the maximum temperature.

24. The method of claim 22 wherein applying pulsed heat comprises pulsing heat between 40 degrees Celsius and 60.5 degrees Celsius.

25. The method of claim 22 wherein applying pulsed heat into the volume of tissue comprises executing a heat cycle including a ramp-up phase, a hold phase, a release phase, and a soak phase.

26. The method of claim 25 wherein the hold phase has a duration between 0.5 seconds to 120 minutes.

27. The method of claim 22 wherein applying pulsed heat comprises applying heat to a continuous surface of tissue having an area of at least 1.27 cm².

28. The method of claim 22 wherein measuring the physiological parameter comprises measuring at least one of blood flow, tissue blood flow, tissue oxygenation, muscle tension, muscle activation, skin temperature, subcutaneous or muscle temperature via direct or inferred measurements, and skin thermal flux.

29. The method of claim 22 wherein measuring the physiological parameter is performed while applying pulsed heat.

30. The method of claim 22, further comprising receiving, at a control device, user preferences including one or more desired physiological parameters.

31. The method of claim 30 wherein defining the treatment parameters comprises defining the treatment parameters based on the user preferences, the treatment parameters comprising temperature and pulse duration, and wherein the method further comprises applying pulsed heat in accordance with the treatment parameters.

32. The method of claim 22 wherein the stimulus device is implanted within the human subject, and wherein applying pulsed heat comprises applying pulsed heat from a location within the human subject.

33. A method of affecting a physiological parameter a human subject to reduce pain of the subject, the method comprising:
applying, via a stimulus device, pulsed heat into a volume of tissue of the human subject, wherein the pulsed heat is configured to affect at least one physiological parameter including blood flow, oxygen delivery, muscle tension, or range of motion;
measuring, via a monitoring device, the at least one physiological parameter of the human subject during application of the pulsed heat; and
defining treatment parameters of the stimulus device such that the pulsed heat synchronizes with the measured physiological parameter to reduce pain.

34. The method of claim 33 wherein applying pulsed heat comprises pulsing heat between 45 degrees Celsius and 60 degrees Celsius.

35. The method of claim 33 wherein applying pulsed heat into the volume of tissue comprises executing a heat cycle including a ramp-up phase, a hold phase, a release phase, and a soak phase.

36. The method of claim 35 wherein the hold phase is less than 3 seconds.

37. The method of claim 33 wherein applying pulsed heat comprises pulsing heat at regular intervals.

38. The method of claim 33 wherein applying pulsed heat comprises pulsing heat at random intervals.

39. The method of claim 33 wherein applying pulsed heat comprises pulsing heat having a temperature of at most 45 degrees Celsius at a rate of at least two pulses per minute.

40. The method of claim 35 wherein the soak time is within a range of one second to 120 minutes.

41. The method of claim 35, further comprising:
receiving, at a control device, user preferences include a number of heat cycles; and
executing the number of heat cycles.

42. The method of claim 35, wherein executing the heat cycle comprises applying heat greater than 45 degrees Celsius, the hold phase having a duration of less than 3 seconds.

43. A method of relaxing muscles and ligaments of a human subject, the method comprising:
applying, via a stimulus device, pulsed heat into a volume of tissue proximate to a joint of the human subject;
measuring, via a stimulus device, a physiological parameter of the human subject; and
defining treatment parameters of the stimulus device such that the pulsed heat synchronizes with the measured physiological parameter to increase a range of motion around the joint.

44. The method of claim 43 wherein the joint is at least one of: a) a major axial join or minor axial joint including at least one of knees, shoulders, hips, ankle, elbow, wrist, fingers, toes, jaw, or areas of the back; b) a joint contained in neuraxial structures of a first cervical vertebrate through a last lumbar vertebra; or c) a sacral joint.

45. The method of claim 43 wherein applying pulsed heat comprises pulsing heat into the volume of tissue prior to a physical activity and/or physical therapy by the human subject.

46. The method of claim 43 wherein measuring the physiological parameter comprises measuring the physiological parameter with at least one of an electromyography or a goniometer.

47. The method of claim 43 wherein the stimulus device is one of a plurality of stimulus devices, and wherein applying pulsed heat comprises applying pulsed heat via the multiple stimulus devices at multiple target sites on the human subject.

48. A method of suppressing an appetite of a human subject, the method comprising:
applying, via a stimulus device, pulsed energy into a volume of tissue including thermoreceptors associated with appetite;
measuring, via a monitoring device, a physiological parameter of the human subject; and
defining treatment parameters of the stimulus device such that the pulsed energy synchronizes with the measured physiological parameter to suppress appetite.

49. The method of claim 48 wherein applying pulsed energy comprises pulsing heat in accordance with the treatment parameters to stimulate and/or induce desensitization of TRPV1 receptors located in skin of the human subject.

50. The method of claim 48 wherein applying pulsed energy comprises pulsing at least one of thermal, vibrational, electrical, and mechanical energy.

51. The method of claim 48 wherein applying pulsed energy comprises pulsing heat between 40 degrees Celsius and 60.5 degrees Celsius.

52. The method of claim 48 wherein applying pulsed energy into the volume of tissue comprises executing a heat cycle including a ramp-up phase, a hold phase, a release phase, and a soak phase.

53. The method of claim 48 wherein the hold phase has a duration of 0.5 seconds to 120 minutes.

54. The method of claim 48 wherein the stimulus device is an implantable device, and wherein applying pulsed energy into the volume of tissue includes applying energy from a target site within the human subject.

55-72. (canceled)
